(19) United States
(12) Patent Application Publication
(10) Pub. No.: US 2026/0158497 A1
(43) Pub. Date: Jun. 11, 2026

(54) METHODS AND SYSTEMS FOR ACHIEVING TEMPERATURE-CONTROLLED REACTION CONDITIONS IN LARGE-VOLUME MICROPLATE CONTAINERS

(71) Applicant: Foundation Medicine, Inc., Boston, MA (US)

(72) Inventor: Charles B. WARREN, Boston, MA (US)

(73) Assignee: Foundation Medicine, Inc., Boston, MA (US)

(21) Appl. No.: 19/356,439

(22) Filed: Oct. 13, 2025

Related U.S. Application Data
(63) Continuation of application No. PCT/US2024/024253, filed on Apr. 12, 2024.
(60) Provisional application No. 63/459,468, filed on Apr. 14, 2023.

(51) Int. Cl. B01L 9/00; B01L 3/00

(52) U.S. Cl. CPC B01L 9/523; B01L 3/50851; B01L 2200/02; B01L 2300/0829; B01L 2300/12; B01L 2300/1805; B01L 2300/1838

(57) ABSTRACT

Embodiments disclosed herein provide exemplary devices, apparatuses, systems, and methods for rapidly achieving temperature-controlled reactions for large-volume microplate containers. For instance, in accordance with embodiments of this disclosure, an adaptor plate (300) is configured to couple a sample plate (250) to a heater (180). The adaptor plate (300) can comprise a receptacle region (310), comprising a plurality of cavities configured to mate with a corresponding well (252) of the sample plate (250). The adaptor plate further comprises an adaptor wall (320) disposed along a perimeter of the receptacle region (310), the adaptor wall (320) configured to contact outer surfaces of one or more wells (252) of the sample plate (250) and further configured to form a series of chambers when the adaptor plate (300) is mated with the sample plate (250). The adaptor plate (300) further comprises a bottom surface (306) configured to mate with the heater (180).

### CROSS-REFERENCE TO RELATED APPLICATION

This application is a continuation application of International Application No. PCT/US 2024/024253, filed internationally on Apr. 12, 2024, which claims priority to and the benefit of U.S. Provisional Ser. No. 63/459,468, filed Apr. 14, 2023, the disclosures of which are herein incorporated by reference in their entirety.

### FIELD OF THE DISCLOSURE

The present disclosure relates generally to a sample plate adapter, and more specifically to a sample plate adapter and associated techniques for achieving temperature controlled reactions for large-volume microplate containers.

### BACKGROUND

In order to analyze DNA from a sample from the individual, the DNA must be extracted from the sample. For liquid-derived diagnostic samples, a preliminary part of the DNA and RNA extraction process includes the removal of interfering protein material by enzymatic digestion. During this process, clarified blood plasma is centrifuged to debulk large debris, combined with significant SDS (sodium dodecyl-sulphate) to partially-solubilize cell membranes, and then Proteinase-K is added and the reaction volume is heated to a target temperature (e.g., 60 C where peak enzymatic activity occurs) and the sample is agitated to promote the extraction process. If during the heating process, the sample is heated to a temperature below the target temperature, the DNA yields of the sample may not be sufficient to conduct an effective analysis of the sample. If during the heating process, the sample is above the target temperature, the sample may result in insufficient digestion due inactivation of the enzyme, and general degradation of the target DNA and RNA in the sample.

One method to heat a sample volume for DNA extraction is to submerge the sample volume in a water bath, where a set point temperature of the water bath is the target temperature of 60 C. While this process can quickly heat the samples and reliably maintain the temperature of the samples at the target temperature, this process is labor intensive and requires a technician to manually perform this process. Additionally, water-bath methods are prone to sample contamination and sample cross-contamination. Moreover, water-bath methods are impractical for use with large microplates.

Automating the process of heating liquid samples for DNA extraction is especially challenging due to the relatively large volume (around 7-10 mL) of patient plasma that comprises each sample for nucleic-acid extraction. Conventional equipment for heating and agitating samples, such as microplates, microplate adaptors and heating devices, are typically designed for much smaller samples, on the order of hundreds of microliters. Additionally, these microplates or sample plates typically include void space between the individual wells. The void space allows heat to be uniformly transferred to each of the wells via the surface area of the wells exposed to the void space.

While large-volume sample plates are available, the large sample volume not only increases the thermal mass needing to be heated, but it reduces or eliminates the void space between sample wells of the sample plate and thus limits the accessible surface area available for moving heat into the fluid volume. Because the sample wells for large-volume sample plates are spaced tightly together, the sample wells located in the interior of the large-volume sample plate do not heat up as quickly as the sample wells located on the perimeter of the sample plate. This non-uniform temperature distribution can impact the DNA extraction yields, making one or more samples of the sample plate unsuitable for genomic analysis. Moreover, adaptors are typically designed to fit only a single corresponding microplate.

Accordingly, there is a need to provide a DNA extractor system with an adaptor plate that can achieve a sufficiently uniform temperature distribution across a large-volume sample plate for DNA extraction.

### BRIEF SUMMARY

Disclosed herein are exemplary devices, apparatuses, systems, and methods for rapidly achieving temperature-controlled reactions for large-volume microplate containers. Embodiments of the present disclosure provide a DNA extractor system may be used to extract DNA and/or perform further analysis of the genomic material extracted from the sample. For example, the DNA extractor system can comprise one or more receptacles (e.g., an adaptor plate) for accommodating one or more sample plates and a heater (e.g., a thermoshaker) for heating and agitating the sample. After the sample plates are loaded, the DNA extractor system may further process and analyze the biological samples in the sample plates. Specifically, embodiments of the present disclosure relate to adaptor plates that can achieve rapid and uniform temperature distribution of the wells in a large-volume sample plate as well as methods of using and designing the same.

Conventional sample plates (e.g., with a volume less than 1 mL) typically include void space between individual wells. An adaptor plate is configured to substantially fill the void space and transfer heat to the wells via the exposed sidewalls of the wells. Because each of the wells is surrounded by void space (e.g., such that sidewalls of the wells are adjacent to the void space), when heat is applied to the sample plate via an adaptor plate, the wells of the sample plate can uniformly increase in temperature (e.g., such that a temperature deviation of the wells is less than 2 C).

In contrast, large-volume sample plates (e.g., with a volume of about 7-10 mL) often lack or have limited void space, which are critical to achieving a uniform temperature distribution across the wells. While commercially available adaptor plates for large sample volume plates may be available, these adaptor plates suffer from two main drawbacks: 1) these adaptor plates are typically designed to be compatible with a single corresponding sample plate and 2) these adaptor plates are unable to achieve a uniform temperature distribution of sample wells.

Embodiments of the present disclosure address these issues and provide a DNA extractor system comprising an adaptor plate that can achieve a uniform temperature distribution across the sample wells in large-volume sample plates. Adaptor plates in accordance with embodiments of the present disclosure are designed to use a combination of convection and conduction to achieve this uniform temperature distribution. Accordingly, embodiments of the present disclosure provide systems and methods to rapidly and uniformly heat a plurality of samples for DNA extraction. Moreover, adaptor plates in accordance with embodiments of the present disclosure may be used with multiple large-volume sample plate designs from multiple commercial sources. Embodiments of the present disclosure can be performed manually (e.g., by a technician) or automatically (e.g., by a robotic system).

Embodiments disclosed herein provide exemplary devices, apparatuses, systems, and methods for rapidly achieving temperature-controlled reactions for large-volume microplate containers. For instance, in accordance with embodiments of this disclosure, an adaptor plate (300) is configured to couple a sample plate (250) to a heater (180), the adaptor plate (300) comprising: a receptacle region (310), the receptacle region comprising a plurality of cavities, at least one cavity (312) of the plurality of cavities configured to mate with a corresponding well (252) of the sample plate (250); an adaptor wall (320) disposed along a perimeter of the receptacle region (310), the adaptor wall (320) comprising: an inner surface (326) configured to contact outer surfaces of one or more wells (252) of the sample plate (250); an outer surface (324); and a top edge (322) connecting the outer surface (324) to the inner surface (326), wherein the top edge (322) and the outer surface (324) of the adaptor wall and a housing (260) of the sample plate (250) are configured to form a series of chambers when the adaptor plate (300) is mated with the sample plate (250); and a bottom surface (306) configured to mate with the heater (180).

In one or more embodiments, the adaptor plate (300) is configured to form a first gap (382) between the top edge (322) of the adaptor wall (320) and the housing (260) of the sample plate (250) when the adaptor plate (300) is mated with the sample plate (250). In one or more embodiments, the adaptor plate (300) is configured to form a second gap (384) between the outer surface (324) of the adaptor wall (320) and an outer wall (262) of the housing (260) of the sample plate (250) when the adaptor plate (300) is mated with the sample plate (250), the second gap different than the first gap. In one or more embodiments, the adaptor wall (320) is configured to transfer heat from the heater (180) to the sample plate (250) via conduction. In one or more embodiments, fluid disposed in the series of chambers is configured to transfer heat from the heater (180) to the sample plate (250) via convection.

In one or more embodiments, the adaptor plate (300) is configured to mate with a second sample plate, the second sample plate different from the first sample plate. In one or more embodiments, the adaptor wall (320) comprises a plurality of slots (314), a slot of the plurality of slots configured to receive a rib (264) of the sample plate (250). In one or more embodiments, the plurality of cavities (312) of the receptacle region (310) are arranged in a grid. In one or more embodiments, a shape of the at least one cavity (312) comprises an inverted pyramid, a hemi-sphere, an inverted cone, or an inverted top-hat profile.

In one or more embodiments, the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder. In one or more embodiments, the adaptor plate (300) is formed by casting, stamping or forging, additive manufacturing, subtractive manufacturing or a combination thereof.

In one or more embodiments, the adaptor plate further comprises: a flange (304) disposed along a foot of the outer surface (324) of the adaptor wall (320), the flange configured to form the series of chambers.

Embodiments of the present disclosure further include a system comprising: a sample plate (250); an adaptor plate (300) configured to mate with the sample plate (250), and a heater (180) configured to mate with the bottom surface (306) of the adaptor plate (300). In one or more embodiments, the adaptor plate (300) comprises: a receptacle region (310), the receptacle region comprising a plurality of cavities, at least one cavity (312) of the plurality of cavities configured to mate with at least one corresponding well (252) of the sample plate (250); an adaptor wall (320) disposed along a perimeter of the receptacle region (310), the adaptor wall (320) comprising: an inner surface (326) configured to contact outer surfaces of one or more wells (252) of the sample plate (250); an outer surface (324); and a top edge (322) connecting the outer surface (324) to the inner surface (326), wherein the top edge (322) and the outer surface (324) of the adaptor wall (320) and a housing (260) of the sample plate (250) are configured to form a series of chambers (370) when the adaptor plate (300) is mated with the sample plate (250); and a bottom surface (306); and a heater (180) configured to mate with the bottom surface (306) of the adaptor plate (300).

In one or more embodiments, the sample plate (250) comprises: the housing (260) having an upper surface (266) and an outer wall (262); a well-region, the well-region comprising a plurality of wells (252) disposed within the housing (260), wherein the at least one corresponding well (252) of the plurality of wells (252) comprises: an opening located at the upper surface (266) of the housing (260); a sidewall (254) extending from the opening into the housing (260); a bottom well-surface (258), the bottom well-surface (258) configured to mate with the at least cavity (312) of the adaptor plate (300); and a plurality of ribs (264), the plurality of ribs (264) disposed within the housing (260) and extending from the sample-well region to the outer wall (262).

In one or more embodiments, the heater comprises a thermoshaker. In one or more embodiments, the adaptor plate (300) is configured to form a first gap between the top edge (322) of the adaptor wall (320) and the housing (260) of the sample plate (250) when the adaptor plate (300) is mated with the sample plate (250). In one or more embodiments, the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap.

In one or more embodiments, the adaptor wall (320) is configured to transfer heat from the heater (180) to the sample plate (250) via conduction. In one or more embodiments, fluid disposed in the series of chambers is configured to transfer heat from the heater (180) to the sample plate (250) via convection. In one or more embodiments, the adaptor plate (300) is configured to mate with a second sample plate, the second sample plate comprising one or more features different from one or more features of the first sample plate. In one or more embodiments, the adaptor wall (320) comprises a plurality of slots (314), a slot of the plurality of slots configured to receive a rib (264) of the sample plate (250). In one or more embodiments, the plurality of cavities (312) of the receptacle region (310) are arranged in a grid. In one or more embodiments, a shape of the at least one cavity (312) comprises an inverted pyramid, a hemi-sphere, an inverted cone, or an inverted top-hat profile.

In one or more embodiments, the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder. In one or more embodiments, the adaptor plate (300) is formed by casting, stamping, forging, additive manufacturing, subtractive manufacturing, or a combination thereof. In one or more embodiments, the adaptor plate further comprising: a flange (304) disposed along a foot of the outer surface (324) of the adaptor wall (320), the flange configured to form the series of chambers.

Embodiments of the present disclosure further comprise a method for coupling a sample plate (250) to a heater (180) via an adaptor plate (300), wherein the adaptor plate (300) comprises: a receptacle region (310), the receptacle region comprising a plurality of cavities (312); an adaptor wall (320) disposed along a perimeter of the receptacle region (310), the adaptor wall (320) comprising: an inner surface (326); a top edge (322); and an outer surface (324) connected to the inner surface (326) by the top edge (322). In one or more embodiments, the method comprises: coupling the adaptor plate (300) to the heater (180); and placing the sample plate (250) on the adaptor plate (300), such that: at least one cavity of the plurality of cavities (312) mates with a corresponding well (252) of the sample plate (250), the inner surface (326) of the adaptor wall (320) mates with outer surfaces of one or more wells (252) of the sample plate (250), and the top edge (322) and the outer surface (324) of the adaptor wall (320) and a housing (260) of the sample plate (250) form a series of chambers when the adaptor plate (300) is mated with the sample plate (250).

In one or more embodiments, coupling the adaptor plate (300) to the heater (180) comprises: placing the adaptor plate (300) on the heater (180); inserting a fastener through an aperture on the adaptor plate (300) and into a corresponding aperture disposed on the heater (180); and securing the adaptor plate (300) to the heater (180) with the fastener. In one or more embodiments, the method further comprises disposing at least one of a thermal paste and a thermal transfer fluid between the adaptor plate (300) and the sample plate (250). In one or more embodiments, the method further comprises disposing at least one of a thermal paste and a thermal transfer fluid between the adaptor plate (300) and the heater (180).

In one or more embodiments, the sample plate (250) comprises: the housing (260) having an upper surface (266) and an outer wall (262); a well-region, the well-region comprising a plurality of wells (252) disposed within the housing (260), wherein the well (252) of the plurality of wells (252) comprises: an opening located at the upper surface (266) of the housing (260); a sidewall (254) extending from the opening into the housing (260); a bottom well-surface (258), the bottom well-surface (258) configured to mate with the at least one cavity (312) of the adaptor plate (300); and a plurality of ribs (264), the plurality of ribs (264) disposed within the housing (260) and extending from the sample-well region to the outer wall (262). In one or more embodiments, the heater comprises a thermoshaker.

In one or more embodiments, the adaptor plate (300) is configured to form a first gap between the top edge (322) of the adaptor wall (320) and the housing (260) of the sample plate (250) when the adaptor plate (300) is mated with the sample plate (250). In one or more embodiments, the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap. In one or more embodiments, the adaptor wall (320) is configured to transfer heat from the heater (180) to the sample plate (250) via conduction. In one or more embodiments, fluid disposed in the series of chambers is configured to transfer heat from the heater (180) to the sample plate (250) via convection. In one or more embodiments, the adaptor plate further comprising: a flange (304) disposed along a foot of the outer surface (324) of the adaptor wall (320), the flange configured to form the series of chambers.

In one or more embodiments, the adaptor plate (300) is configured to mate with a second sample plate, the second sample plate comprising one or more features different from one or more features of the first sample plate. In one or more embodiments, the adaptor wall (320) comprises a plurality of slots (314), a slot of the plurality of slots configured to receive a rib (264) of the sample plate (250). In one or embodiments, the plurality of cavities (312) of the receptacle region (310) are arranged in a grid. In one or more embodiments, a shape of the at least one cavity (312) comprises an inverted pyramid, a hemi-sphere, an inverted cone, or an inverted top-hat profile.

In one or more embodiments, the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder. In one or more embodiments, the adaptor plate (300) is formed by casting, stamping, forging, additive manufacturing, subtractive manufacturing, or a combination thereof.

Embodiments of the present disclosure further comprise methods for heating a sample. In some embodiments, the method comprises: loading the sample into a plurality of wells (252) of a sample plate (250); mounting an adaptor plate (300) to a heater (180), wherein the adaptor plate (300) comprises: a receptacle region (310), the receptacle region comprising a plurality of cavities (312); an adaptor wall (320) disposed along a perimeter of the receptacle region (310), the adaptor wall (320) comprising: an inner surface (326); an outer surface (324); and a top edge (322) connecting the outer surface (324) to the inner surface (326); placing the sample plate (250) on the adaptor plate (300) such that: at least one cavity of the plurality of cavities (312) mates with a corresponding well (252) of the sample plate (250), the inner surface (326) of the adaptor wall (320) mates with outer surfaces of one or more wells (252) of the sample plate (250), and the top edge (322) and the outer surface (324) of the adaptor wall and a housing (260) of the sample plate (250) form a series of chambers when the adaptor plate (300) is mated with the sample plate (250); operating the heater at a first temperature for a first time period; and operating the heater at a second temperature for a second time period. In one or more embodiments, the adaptor plate further comprises a flange (304) disposed along a foot of the outer surface (324) of the adaptor wall (320), the flange configured to form the series of chambers.

In one or more embodiments, the method further comprises heating the sample plate (250) by conduction, via the adaptor wall (320) and further via the plurality of cavities (312) of the adaptor plate (300). In one or more embodiments, the method further comprises heating the sample plate (250), by convection, via a fluid disposed in the series of chambers. In one or more embodiments, the combination of convective and conductive heating provides a uniform temperature distribution of the sample disposed in the plurality of wells (252). In one or more embodiments, the uniform temperature distribution comprises a temperature deviation between wells of the plurality of wells (252) of about two degrees centigrade.

In one or more embodiments, the sample plate (250) comprises: the housing (260) having an upper surface (266) and an outer wall (262); a well-region, the well-region comprising a plurality of wells (252) disposed within the housing (260), wherein the well (252) of the plurality of wells (252) comprises: an opening located at the upper surface (266) of the housing (260); a sidewall (254) extending from the opening into the housing (260); a bottom well-surface (258), the bottom well-surface (258) configured to mate with the at least one cavity (312) of the adaptor plate (300); and a plurality of ribs (264), the plurality of ribs (264) disposed within the housing (260) and extending from the sample-well region to the outer wall (262). In one or more embodiments, the heater comprises a thermoshaker.

In one or more embodiments, the adaptor plate (300) is configured to form a first gap between the top edge (322) of the adaptor wall (320) and the housing (260) of the sample plate (250) when the adaptor plate (300) is mated with the sample plate (250). In one or more embodiments, the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap. In one or more embodiments, the adaptor wall (320) is configured to transfer heat from the heater (180) to the sample plate (250) via conduction. In one or more embodiments, fluid disposed in the series of chambers is configured to transfer heat from the heater (180) to the sample plate (250) via convection. In one or more embodiments, the adaptor plate (300) is configured to mate with a second sample plate, the second sample plate comprising one or more features different from one or more features of the first sample plate.

In one or more embodiments, the adaptor wall (320) comprises a plurality of slots (314), a slot of the plurality of slots configured to receive a rib (264) of the sample plate (250). In one or more embodiments, the plurality of cavities (312) of the receptacle region (310) are arranged in a grid. In one or more embodiments, a shape of the at least one cavity (312) comprises an inverted pyramid, a hemi-sphere, an inverted cone, or an inverted top-hat profile.

In one or more embodiments, the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder. In one or more embodiments, the adaptor plate (300) is formed by casting, stamping or forging, additive manufacturing, subtractive manufacturing or a combination thereof.

Embodiments of the present disclosure further comprise methods for measuring a temperature of wells (252) of a sample plate (250). In one or more examples, the method comprises: loading a sample into a plurality of wells (252) of the sample plate (250); mounting an adaptor plate (300) to a heater (180), wherein the adaptor plate comprises: a receptacle region (310), the receptacle region comprising a plurality of cavities (312), an adaptor wall (320) disposed along a perimeter of the receptacle region (310), the adaptor wall (320) comprising: an inner surface (326), an outer surface (324), and a top edge (322) connecting the outer surface (324) to the inner surface (326); placing the sample plate (250) on the adaptor plate (300) such that: at least one cavity of the plurality of cavities (312) mates with a corresponding well (252) of the sample plate (250), the inner surface (326) of the adaptor wall (320) mates with outer surfaces of one or more wells (252) of the sample plate (250), and the top edge (322) and the outer surface (324) of the adaptor wall and a housing (260) of the sample plate (250) form a series of chambers when the adaptor plate (300) is mated with the sample plate (250); disposing a temperature sensor in a first well (252) of the sample plate; sealing the plurality of wells; operating the heater at a first temperature for a first time period; operating the heater at a second temperature for a second time period; and receiving measurements from the temperature sensor during the first time period and the second time period. In one or more embodiments, the method further comprises disposing a second temperature sensor in a second well of the sample plate (250).

Embodiments of the present disclosure further comprise a DNA extractor. In one or more embodiments, the DNA extractor comprises: a sample plate (250); an adaptor plate (300) configured to mate with the sample plate (250), and a heater (180) configured to mate with the bottom surface (306) of the adaptor plate (300). In such embodiments, the adaptor plate (300) comprises a receptacle region (310), the receptacle region comprising a plurality of cavities, at least one cavity (312) of the plurality of cavities configured to mate with a corresponding well (252) of the sample plate (250); an adaptor wall (320) disposed along a perimeter of the receptacle region (310), the adaptor wall (320) comprising: an inner surface (326) configured to contact outer surfaces of one or more wells (252) of the sample plate (250), an outer surface (324); and a top edge (322) connecting the outer surface (324) to the inner surface (326), wherein the top edge (322) and the outer surface (324) of the adaptor wall and a housing (260) of the sample plate (250) are configured to form a series of chambers when the adaptor plate (300) is mated with the sample plate (250); and a bottom surface (306).

Embodiments of the present disclosure further comprise a method for extracting deoxyribonucleic acid (DNA) molecules from a sample from a subject using a DNA extraction device. In one or more embodiments, the method comprises: providing the sample in a sample plate (250); mounting an adaptor plate (300) to a heater (180); placing the sample plate (250) on the adaptor plate (300) such that: at least one cavity of the plurality of cavities (312) mates with a corresponding well (252) of the sample plate (250), the inner surface (326) of the adaptor wall (320) mates with outer surfaces of one or more wells (252) of the sample plate (250), and the top edge (322), the outer surface (324) of the adaptor wall, and a housing of the sample plate form a series of chambers when the adaptor plate (300) is mated with the sample plate (250); heating, via the heater (180), the sample in the sample plate (250); extracting a plurality of nucleic acid molecules, including at least one DNA molecule, from the sample in the sample plate (250); ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; and sequencing, by a sequencer, the captured nucleic acid molecules to obtain a plurality of sequence reads that represent the captured nucleic acid molecules, wherein one or more of the plurality of sequencing reads overlap one or more gene loci within a subgenomic interval in the sample. In such embodiments, the adaptor plate comprises: a receptacle region (310), the receptacle region comprising a plurality of cavities (312); an adaptor wall (320) disposed along a perimeter of the receptacle region (310), the adaptor wall (320) comprising: an inner surface (326); an outer surface (324); and a top edge (322) connecting the outer surface (324) to the inner surface (326).

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A depicts an exploded view of a non-limiting example of an exemplary DNA extractor system, in accordance with some embodiments.

FIG. 1B depicts a cross-sectional view of a non-limiting example of an exemplary DNA extractor system, in accordance with some embodiments.

FIG. 2A depicts a bottom-up perspective view of a non-limiting example of an exemplary sample plate, in accordance with some embodiments.

FIG. 2B depicts a detail view of a non-limiting example of an exemplary sample plate, in accordance with some embodiments.

FIG. 2C depicts a top-down perspective view of a non-limiting example of an exemplary sample plate, in accordance with some embodiments.

FIG. 2D depicts a cross-sectional view of a non-limiting example of an exemplary sample plate, in accordance with some embodiments.

FIG. 3A depicts a perspective view of a non-limiting example of an exemplary adaptor plate, in accordance with some embodiments.

FIG. 3B depicts a top view of a non-limiting example of an exemplary adaptor plate, in accordance with some embodiments.

FIG. 3C depicts a side view of a non-limiting example of an exemplary adaptor plate, in accordance with some embodiments.

FIG. 3D depicts a side view of a non-limiting example of an exemplary adaptor plate, in accordance with some embodiments.

FIG. 4A depicts a perspective view of a non-limiting example of an exemplary adaptor plate and an exemplary sample plate, in accordance with some embodiments.

FIG. 4B depicts an exploded view of a non-limiting example of an exemplary adaptor plate and an exemplary sample plate, in accordance with some embodiments.

FIG. 4C depicts a cross-sectional view of a non-limiting example of an exemplary adaptor plate and an exemplary sample plate, in accordance with some embodiments.

FIG. 5 illustrates an exemplary process for mounting a sample plate to an adaptor plate, in accordance with some embodiments.

FIG. 6 illustrates an exemplary process for heating a sample plate via an adaptor plate, in accordance with some embodiments.

FIG. 7 illustrates an exemplary plot of the temperature of exemplary adaptor plates and a heater over time when heated, in accordance with some embodiments.

FIG. 8 illustrates an exemplary process for measuring one or more samples in a sample plate, in accordance with some embodiments.

FIG. 9 depicts an exemplary system for measuring a temperature in a plurality of wells, in accordance with some embodiments.

FIG. 10 illustrates an exemplary process for extracting DNA from one or more samples, in accordance with some embodiments.

FIG. 11A depicts an exemplary plot illustrating the DNA extraction yields for samples heated in accordance with some embodiments.

FIG. 11B illustrates an exemplary plot of the temperature of exemplary samples over time, in accordance with some embodiments.

FIG. 12A depicts a perspective view of an exemplary sample microplate and adaptor.

FIG. 12B depicts a cross-sectional view of an exemplary sample microplate and adaptor.

FIG. 13 depicts a cross-sectional view of an exemplary sample microplate and adaptor.

### DETAILED DESCRIPTION

Disclosed herein are exemplary devices, apparatuses, systems, and methods for rapidly achieving temperature-controlled reactions for large-volume microplate containers. Embodiments of the present disclosure provide a DNA extractor system may be used to extract DNA and/or perform further analysis of the genomic material extracted from the sample. For example, the DNA extractor system can comprise one or more receptacles (e.g., an adaptor plate) for accommodating one or more sample plates and a heater (e.g., a thermoshaker) for heating and agitating the sample. After the sample plates are loaded, the DNA extractor system may further process and analyze the biological samples in the sample plates. Specifically, embodiments of the present disclosure relate to adaptor plates that can achieve rapid and uniform temperature distribution of the wells in a large-volume sample plate as well as methods of using and designing the same.

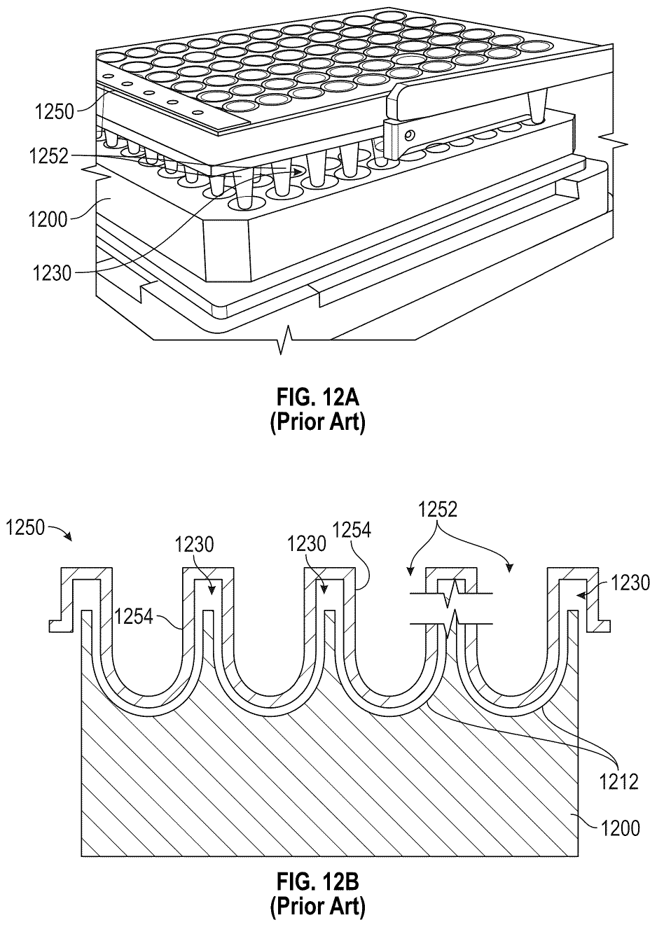

FIGS. 12A and 12B, illustrate conventional sample plate 1250 (e.g., with a volume less than 1 mL) and a corresponding adaptor plate 1200. As shown in the figure, the sample plate 1250 includes a plurality of wells 1252 and void space 1230 located between the individual wells 1252. An adaptor plate, such as adaptor plate 1200 is configured to substantially fill the void space 1230 and transfer heat to each of the wells 1252 via the exposed sidewalls 1254 of the wells. For instance, referring specifically to FIG. 12B, the adaptor plate 1200 includes a plurality of cavities 1212 that interface with the sidewalls 1254 of the sample plate 1250 to facilitate heat transfer. Because each of the wells 1252 is surrounded by void space 1230 (e.g., such that sidewalls 1254 of the wells 1252 are adjacent to the void space 1230), when heat is applied to the sample plate 1250 via the adaptor plate 1250, the wells 1252 of the sample plate 1250 will uniformly increase in temperature (e.g., such that a temperature deviation of the wells is less than 2 C).

In contrast, large-volume sample plates (e.g., with a volume of about 7-10 mL) often lack or have limited void spaces that are critical to achieving a uniform temperature distribution across the wells. While commercially available adaptor plates for large sample volume plates may be available, these adaptor plates are typically designed to be compatible with a single corresponding sample plate and are unable to achieve a uniform temperature distribution of sample wells (e.g., such that the temperature of each of the wells on the sample plate are within less than 2 C of each other).

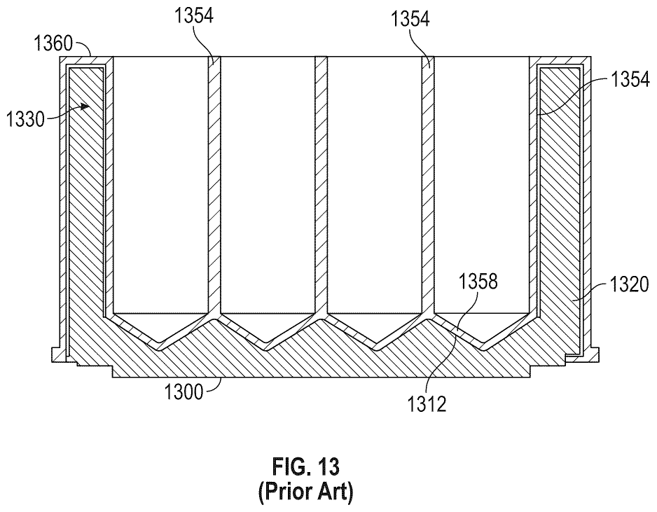

FIG. 13 illustrates an embodiment of a conventional sample plate 1350 and a corresponding adaptor plate 1300. Sample plate 1350 corresponds to a large volume sample plate that lacks void space between the individual wells 1352. For instance, one or more wells 1352 share a sidewall 1354. Due to the lack of void space between the individual wells, the adaptor plate 1300 (e.g., the adaptor wall 1320 and one or more cavities 1312) may mate with the perimeter the plurality of wells 1352 of the sample plate 1350. As shown in the figure, the adaptor plate is configured to substantially fill any void space 1330 within housing 1360 of the sample plate 1350, but does not fill any void space between the plurality of wells 1352. Because the wells located on the perimeter of the plurality of wells 1352 receive heat via a bottom surface of the well 1328 and one or more sidewalls 1354, these outer wells will rise in temperature more quickly than inner wells that receive heat via just the bottom surface 1328. This non-uniform heating results in thermal edge effects, whereby outer wells (e.g., wells disposed on a perimeter of the sample plate) heat more quickly than inner wells of the sample plate. In some cases the thermal edge effects can result in temperature variation between outer and inner wells of 5-10 C.

Accordingly, embodiments of the present disclosure provide a DNA extractor system comprising an adaptor plate that can achieve a uniform temperature distribution across the sample wells large-volume sample plates. Specifically, the adaptor plate was designed to minimize thermal edge effects, whereby outer wells (e.g., wells disposed on a perimeter of the sample plate) heat more quickly than inner wells of the sample plate. Adaptor plates in accordance with embodiments of the present disclosure are designed to use a combination of convection and conduction to achieve this uniform temperature distribution. Accordingly, embodiments of the present disclosure provide systems and methods to rapidly and uniformly heat a plurality of samples for DNA extraction. Moreover, adaptor plates in accordance with embodiments of the present disclosure may be used with multiple large-volume sample plate designs from multiple commercial sources. Embodiments of the present disclosure can be performed manually (e.g., by a technician) or automatically (e.g., by a robotic system).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “uniform temperature distribution” as applied to a description of a sample plate having a plurality of wells with a “uniform temperature distribution” is used to mean that the temperatures associated with each of the wells of the sample plate have a deviation of 0 to 2 C.

### DNA Extractor System

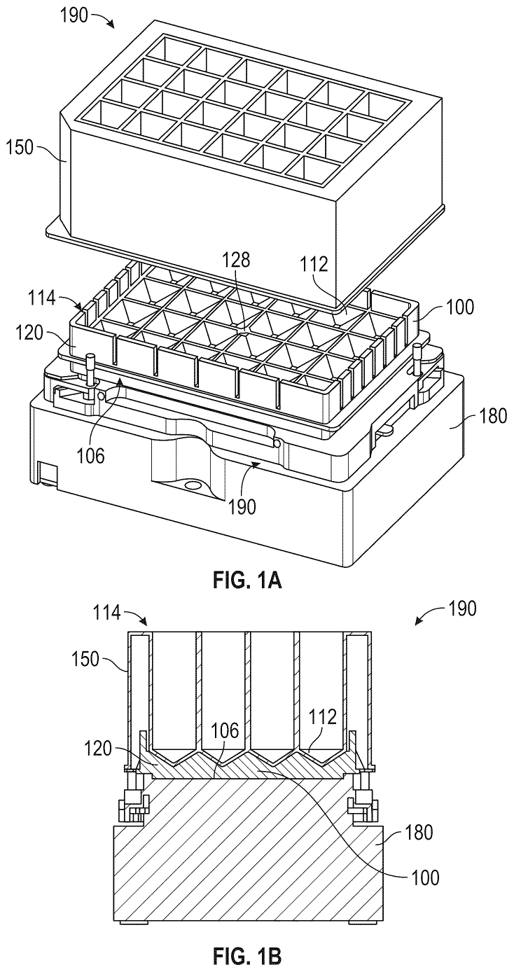

FIGS. 1A and 1B illustrate an exemplary DNA extractor system 190 comprising an adaptor plate 100, a sample plate 150, and a heater 180 in accordance with embodiments of this disclosure. In one or more embodiments, the DNA extractor system 190 can be used to extract DNA from one or more samples from a patient. For instance, a preliminary part of the DNA and RNA extraction process for liquid-derived diagnostic samples includes the removal of interfering protein material by enzymatic digestion. During this process, clarified blood plasma is centrifuged to debulk large debris, combined with significant SDS (sodium dodecyl-sulphate) to partially-solubilize cell membranes, and then Proteinase-K is added and the reaction volume is heated to a target temperature (e.g., 60 C where peak enzymatic activity occurs) and the sample is agitated to promote the extraction process. As shown in the figure, the sample plate 150 can be placed on the adaptor plate 100. The adaptor plate 100 may be mounted on and/or secured to the heater 180. In some embodiments, the heater can heat and agitate (e.g., shakes) the sample.

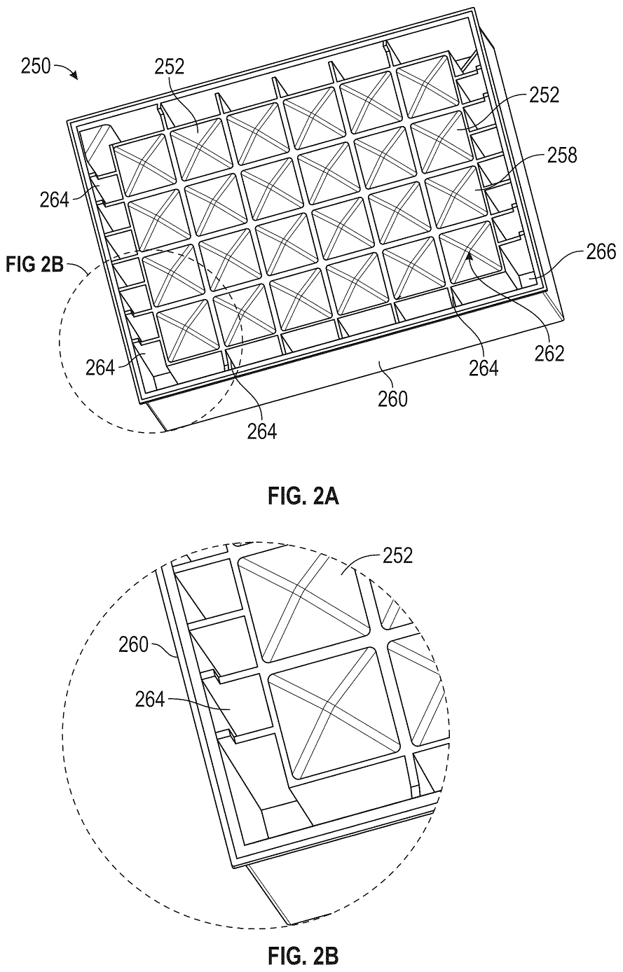

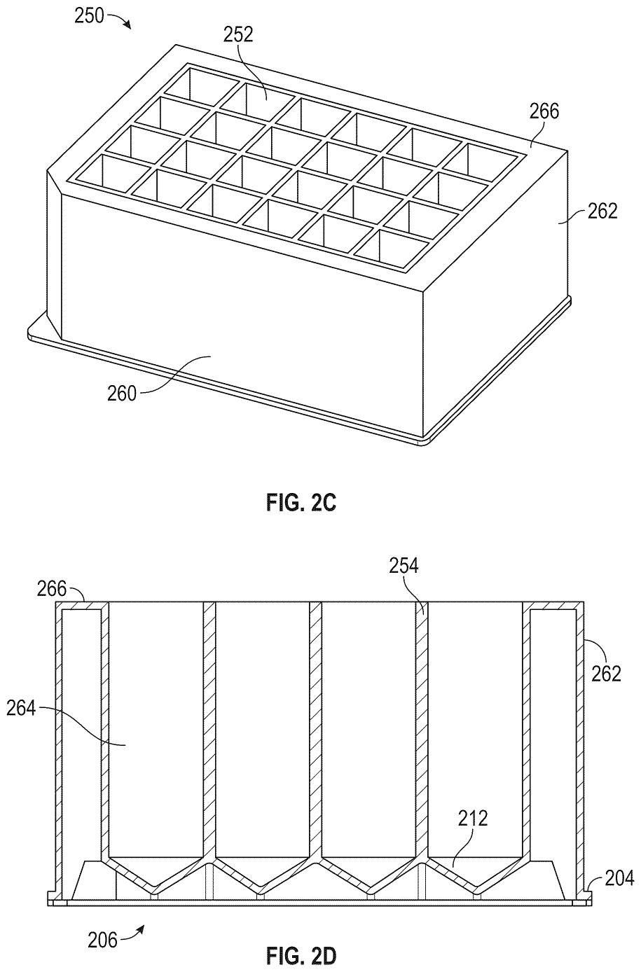

In one or more examples, sample plate 150 can correspond to a large-volume sample plate, with a well volume of 10 mL. In some embodiments, the sample plate can correspond to a large-volume sample plate, with a well volume of 7-11 mL. FIGS. 2A-2D illustrates a non-limiting example of a sample plate 250 according to embodiments of this disclosure. In one or more example, sample plate 250 may correspond to sample plate 150. For instance, sample plate 250 can correspond to a large-volume sample plate, where a capacity of the sample wells of the sample plate 250 is about 7-10 mL. As shown in the figure, the sample plate 250 can comprise a housing 260, a well-region comprising a plurality of wells 252, and a plurality of ribs 264. In some embodiments, the housing 260 includes an upper surface 266 that connects the well-region to an outer wall 262.

In some embodiments, sample plate 250 can further include a well-region that includes a plurality of wells 252 disposed within the housing 260. As shown in the figure, the plurality of wells 252 may be arranged in a grid. Referring to FIGS. 2C and 2D the wells 252 can include an opening located at the upper surface 266 of the housing 260. That is, the upper surface may comprise one or more openings, each opening corresponding a well 252. Each well 252 may further comprise one or more sidewalls 254 that extend from the opening into the housing 260. Each well 252 may further comprise a bottom well-surface 258. As shown in FIGS. 2A, 2B, and 2D the bottom well-surface 258 comprises an inverted pyramid shape configured to interface or mate with at least one cavity of the adaptor plate (e.g., cavity 112 of the adaptor plate 100). A skilled artisan will appreciate that the shape of the bottom well-surface of the sample plate may comprise other shapes, including but not limited to, a hemi-sphere, an inverted cone, or an inverted top-hat profile.

The sample plate 250 can further comprise a plurality of ribs 264, the plurality of ribs 264 disposed within the housing 260 and extending from the sample-well region to the outer wall 262.

Due to the large-volume capacity of the wells 252, void space between the plurality of wells may be limited or non-existent. As discussed above, the lack of void space between the plurality of wells limits the surface area available for heating wells that are located in an interior (e.g., such that the interior wells do not have a side wall 254 along a perimeter of the well region) of the well-region. For instance, the sample plate may be heated, via conduction by an adaptor plate (e.g., adaptor plate 100), via the bottom well-surface 258 and the sidewalls 254 that are located along the perimeter of the well region. Because outer-wells (e.g., outer well 2520 in FIG. 2D) located along a perimeter of the well region may receive conductive heat from the adaptor plate via multiple surfaces, these wells may rise in temperature more quickly than inner-wells (e.g., 252i in FIG. 2D). This results in a non-uniform temperature distribution across the wells that can result in sub-optimal DNA extraction yields for the samples (e.g., via overheating or underheating). Accordingly, embodiments of the present disclosure provide an adaptor plate for a DNA extractor system that can provide a uniform temperature distribution for large-volume adaptor plates.

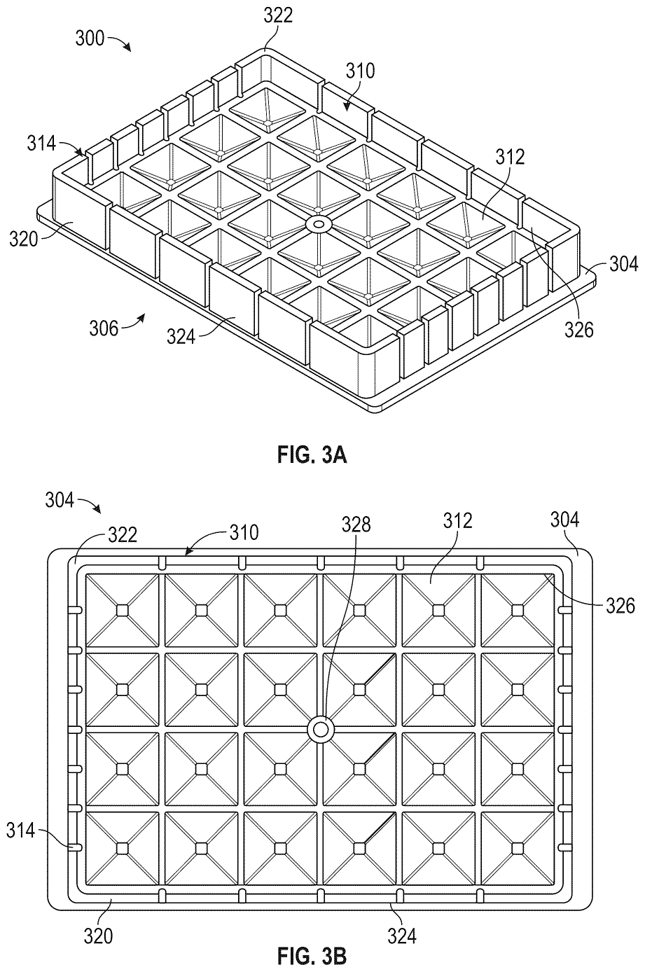

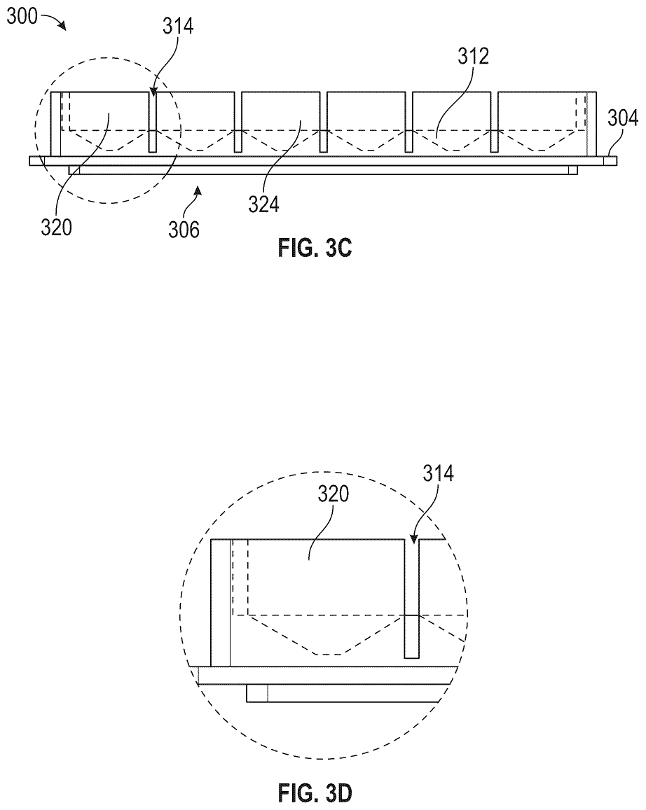

FIGS. 3A-3D illustrate an exemplary adaptor plate 300 of an exemplary DNA extractor system (e.g., DNA extractor system 190 of FIGS. 1A and 1B) for rapidly and uniformly heating a sample plate according to embodiments of this disclosure. As discussed above, the adaptor plate can be configured to receive a sample plate (e.g., sample plate 250 of FIGS. 2A-2D), such as a large-well sample plate having a sample well capacity of about 7-10 mL. The adaptor plate 300 can further be configured to mount to a heater (e.g., heater 180 of FIGS. 1A and 1B) for heating one or more samples disposed in the sample plate. In one or more embodiments, the heater may comprise a bioshaker or thermoshaker for heating and agitating samples disposed in the sample plate.

With reference to FIG. 3A, the adaptor plate 300 can comprise a receptacle region 310 configured to mate with a sample plate, an adaptor wall 320 disposed along a perimeter of the receptacle region 310, and a bottom surface 306 configured to mate with a heater (e.g., thermoshaker). As shown in the figure, the receptacle region 310 comprises a plurality of cavities 312. The geometry of the plurality of cavities 312 is dimensioned to receive and mate with a bottom surface (e.g., bottom surface 258) of the sample plate (e.g., sample plate 250). In some examples, the plurality of cavities 312 can be arranged in a grid. The cavities 312 are depicted as having an inverted-pyramid geometry. However, a skilled artisan will understand that the shape of the cavity may vary based on a corresponding sample plate. For instance, the shape of the cavities may comprise, but is not limited to, an inverted pyramid, a hemi-sphere, an inverted cone, or an inverted top-hat profile.

The adaptor wall 320 is disposed along a perimeter of the receptacle region 310 and is configured to interface with an outer surface of the sample wells of a sample plate. As shown in the figure, the adaptor wall 320 comprises an inner surface 326 configured to contact outer surfaces of one or more wells of a sample plate. The adaptor wall 320 further comprises an outer surface 324. The adaptor wall 320 further comprises a top edge 322 connecting the outer surface 324 to the inner surface 326. The adaptor wall 320 may further comprise a plurality of slots 314 for receiving a plurality of corresponding ribs (e.g., ribs 264) of a sample plate (e.g., sample plate 250). The adaptor plate 300 further comprises a flange 304 disposed along a foot of the outer surface 324 of the adaptor wall 320.

In one or more embodiments, the receptacle region can comprise an aperture 328. The aperture can be configured to receive one or more fasteners for securing the adaptor plate 300 to a heater. Referring, to FIGS. 3C and 3D, a bottom surface 306 of the adaptor plate 300 may not correspond to (e.g., may not be parallel to or is otherwise independent of) the geometry of an upper surface of the adaptor plate. For example, while an upper surface of the adaptor plate 300, e.g., corresponding to the receptacle region 310, may include a plurality of cavities 312 to mate with a sample plate, the bottom surface 306 may be configured to mate with a heater (e.g., heater 180). As shown in these figures a profile of the bottom surface 306 may be include one or more steps. A skilled artisan will understand that the geometry of the bottom surface 306 may depend on the geometry of the corresponding heater. In one or more examples, the geometry of the bottom surface 306 may be configured to mate with one or more models of heaters.

In one or more embodiments, the adaptor plate 300 can be formed from any material that efficiently conducts heat such as, but not limited to, aluminum, copper, silver, gold. In some embodiments, the adaptor plate 300 can be formed from an alloy of aluminum, copper, silver, or gold. In some embodiments, the adaptor plate 300 can be formed from a material comprising aluminum, copper, silver, or gold compounded with a binder or filler in an extruded filament. In some embodiments, the adaptor plate 300 can be formed from a material comprising aluminum, copper, silver, or gold with a binder or carrier as a powder for metal-sintering manufacturing. In one or more embodiments, the adaptor plate 300 may be formed by casting, stamping or forging, additive manufacturing (e.g., including but not limited to fused-deposition modeling, selective-layer-sintering), subtractive manufacturing (e.g., including but not limited to CNC milling, electrical-discharge machining) or a combination thereof.

Referring back to FIGS. 1A and 1B, the heater 180 may be configured to heat and/or agitate one or more samples disposed in the sample plate 150. In one or more examples, the heater 180 may be a bioshaker or thermoshaker. In one or more examples the heater may be configured to have a heating set point up to 99 C with an accuracy of 0.1 C and a hot plate uniformity of 0.5 C. In one or more examples, the heater may be capable of mechanically agitating the samples in the sample place and have a 3 mm orbital mixing frequency of 200 to 2000 rpm and a mechanical sizing to accommodate large-volume sample plates.

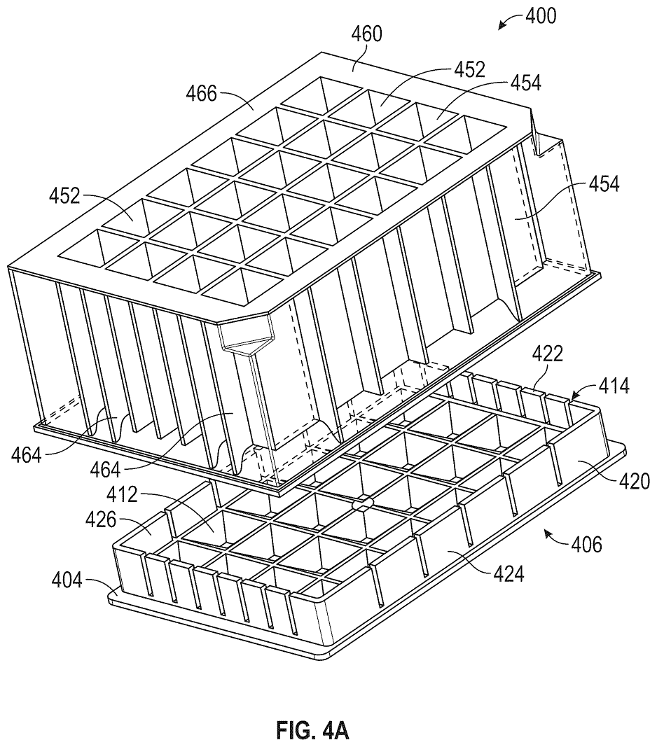

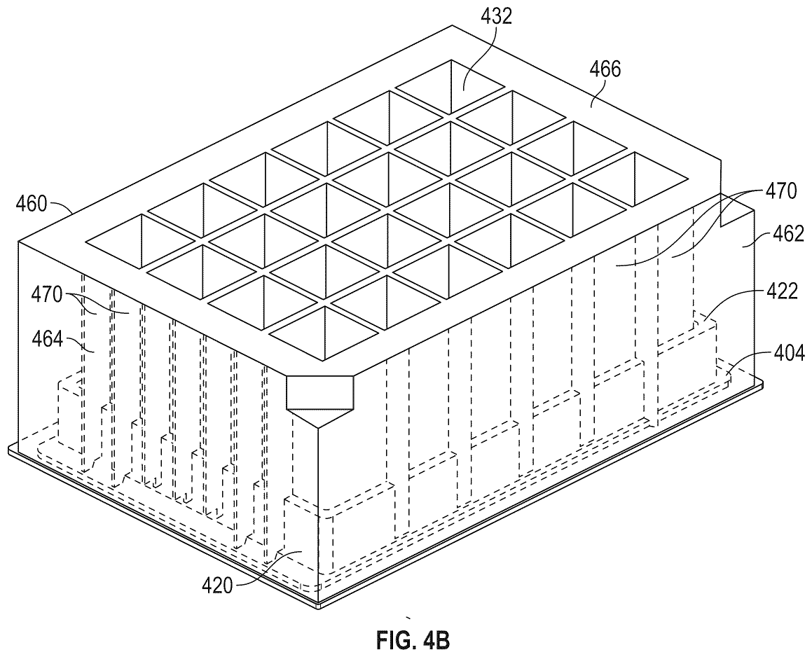

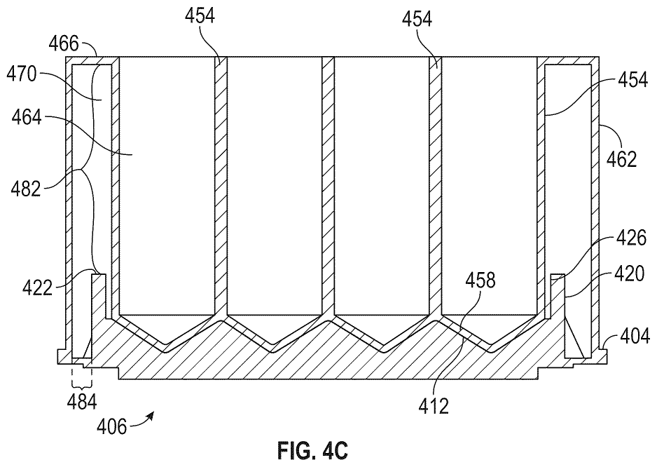

In one or more examples, the heater 180 can be configured to mate with a bottom surface 106 of the adaptor plate. For instance, the dimensions and shape of the bottom surface 106 may correspond to a mounting surface 182 of the heater 180. As shown in FIG. 4B, the mounting surface 182 may be substantially flat with an outer stepped portion 184. The bottom surface 106 of the adaptor plate 100 may include a corresponding stepped feature. In one or more examples, the mounting surface 182 may comprise an aperture (not shown) that corresponds to an aperture 128 of the adaptor plate 100. In one or more examples, a fastener (e.g., a screw, a bolt, etc.) may be used to affix or secure the adaptor plate to the heater 180. In one or more examples, thermal paste and/or thermal transfer fluid (e.g., silicone, mineral oil, etc.) may be disposed between the bottom surface 106 of the adaptor plate 100 and the mounting surface 182 of the heater 180 to facilitate heat transfer between the two components.

FIGS. 4A-4C illustrate a DNA extractor system comprising an adaptor plate 400 and a sample plate 450 in accordance with embodiments of this disclosure. In one or more examples, adaptor plate 400 and sample plate 450 can correspond to one or more of the adaptor plates and sample plates described above. FIG. 4A illustrates an exploded view of an adaptor plate 400 and sample plate 450. For instance, adaptor plate 400 can comprise a receptacle region 410 configured to mate with a sample plate, an adaptor wall 420 disposed along a perimeter of the receptacle region 410, and a bottom surface 406 configured to mate with a heater (e.g., heater 180). As shown in the figure, the receptacle region 410 comprises a plurality of cavities 412. The geometry of the plurality of cavities 412 is dimensioned to receive and mate with a bottom surface of the sample plate. In some examples, the plurality of cavities can be arranged in a grid. The cavities 412 are depicted as having an inverted-pyramid geometry. However, a skilled artisan will understand that the shape of the cavity may vary based on a corresponding sample plate. For instance, the shape of the cavities may comprise, but is not limited to, an inverted pyramid, a hemi-sphere, an inverted cone, or an inverted top-hat profile.

The adaptor wall 420 is disposed along a perimeter of the receptacle region 410 and is configured to mate with an outer surface of the sample wells of a sample plate. As shown in the figure, the adaptor wall 420 comprises an inner surface 426 configured to contact outer surfaces of one or more wells 452 of the sample plate 450. The adaptor wall 420 further comprises an outer surface 424. The adaptor wall 420 further comprises a top edge 422 connecting the outer surface 424 to the inner surface 426. The adaptor wall 420 may further comprise a plurality of slots 414 for receiving a plurality of corresponding ribs 464 of a sample plate 450. The adaptor plate 400 further comprises a flange 404 disposed along a foot of the outer surface 424 of the adaptor wall 420.

In one or more examples, sample plate 450 of FIGS. 4A-3C can correspond to sample plate 250. For instance, sample plate 450 can comprise a housing 460, a well-region comprising a plurality of wells 452, and a plurality of ribs 464. In some embodiments, the housing 460 includes an upper surface 466 that connects the outer wall 462 to the well region. The well-22 region includes a plurality of wells 452 disposed within the housing 460. The plurality of wells can comprise one or more sidewalls 454 and a bottom surface 458. As shown in FIG. 4C, the bottom well-surface 458 may comprise an inverted pyramid shape that is configured to interface with at least one cavity 412 of the adaptor plate 400. The sample plate 450 can comprise further comprise a plurality of ribs 464, the plurality of ribs 464 disposed within the housing 460 and extending from the sample well region to the outer wall 462. As shown in FIG. 4B, the plurality of ribs 464 may be received in a corresponding slot 414 of the adaptor plate 400.

In one or more embodiments, the adaptor plate 400 can be designed to interface or mate with a second sample plate that is different than sample plate 250. For instance, the second sample plate may have a different dimensions and or locations of one or more features, including but not limited to well size, well height, rib size, rib location, and the like. In some instances, the second sample plate may differ from the first sample plate due to tolerance variations between the sample plate 450 and the second sample plate.

FIGS. 4B and 4C illustrate exemplary views of a sample plate 450 placed on or mounted to an adaptor plate 400 such that the sample plate is mated with the adaptor plate 400. As used herein, the term “mated” may be used to describe such that the one or more surfaces of the sample plate and adaptor plate are in contact or in close proximity such that conductive heat transfer occurs between these components. FIG. 4C corresponds to a cross-sectional view of taken from line 4C shown in FIG. 4B. Referring to FIG. 4C, when the sample plate 450 is placed on the adaptor plate 400, the one or more cavities 412 of the adaptor plate 400 can mate with a bottom surface 458 of a corresponding well 452. Additionally, an inner surface 426 of the adaptor wall 420 can mate with at least a portion of a sidewall 454 of a well 452 located on a perimeter of the well region of the sample plate 450. In one or more examples, mating the one or more cavities 412 to the bottom surface 458 of a corresponding well and/or mating the inner surface 426 of the adaptor wall 420 with at least a portion of a sidewall 454 of a well 452 located on a perimeter of the well region may facilitate conductive heat transfer between the adaptor plate 400 and the sample plate 450. To the extent that FIG. 4C shows a clearance between the sidewall 454 and the inner surface 426 of the adaptor wall 420, a skilled artisan would understand that the clearance may be small (e.g., less than 0.2 mm or in some embodiments, less than 0.5 mm) such that conductive heat transfer can occur between these two surfaces. In one or more examples, a thermal paste and/or a thermal transfer fluid may be applied between the sample plate 450 and the adaptor plate 400 to improve conduction between these components when mated. In such examples, the thermal paste and/or thermal transfer fluid may fill any clearances between the sample plate 450 and the adaptor plate 400.

As shown in FIGS. 4B and 4C, a series of chambers 470 may be formed between the adaptor plate 400 and the sample plate 450 when these components are mated. In one or more embodiments, a fluid (e.g., air) may be disposed in the chambers 470. In such embodiments, the fluid in the chambers 470 may heat the plurality of wells 452 via convective heat transfer. In one or more examples, a chamber of the series of chambers 470 may be bounded by a pair of ribs 464, the upper surface 466 and an outer surface 462 of the housing 460 and the top edge 422 and the outer surface 424 of the adaptor wall 120 and the foot 404 of the adaptor plate 400.

For instance, each chamber 470 may be formed laterally between a pair of ribs 464 of the sample plate. FIG. 4C, illustrates a cross-sectional view of a chamber 470. As shown in the figure, the plurality of chambers 470 may be formed because the adaptor wall is sized such that it does not fill the void between the sidewall 454 of the one or more wells 452 and the outer wall 462 of the housing. For instance, as shown in the figure, the height of the adaptor wall 420 may be less than a height of a sidewall 454. In one or more examples, the height of the adaptor wall may be less than half the height of the sidewall. In one or more examples, the height of the adaptor wall may be less than a quarter of the height of the sidewall. This height differential forms a vertical gap 482 between the top edge 422 of the adaptor wall 420 and the upper surface 466 of the housing 460 of the sample plate 450 when the adaptor plate 400 is mated with the sample plate 450. Additionally, the width of the adaptor wall 420 may be less than the horizontal distance between the perimeter sidewall 454 of a well and the outer wall 462 of the housing 460 of the sample plate. In some embodiments, the width of the adaptor wall 420 may be less than half the horizontal distance between the perimeter sidewall 454 of a well and the outer wall 462 of the housing 460 of the sample plate. This may form a horizontal gap 484 between the outer surface 424 of the adaptor wall 420 and the outer wall 462 of the housing 460 of the sample plate 450 when the adaptor plate 400 is mated with the sample plate 450. As shown in the figure, the vertical gap 482 may be different from the horizontal gap.

Compared to conventional adaptor plates (e.g., adaptor plate1300), embodiments of the present disclosure provide more uniform heating across the wells of the sample plate (e.g., sample plate 150), particularly, large-volume sample plates. For instance, FIG. 13 illustrates a conventional adaptor plate 1300 for a large-volume sample plate 1350. As shown in the figure, the adaptor plate substantially fills any void space present in the housing 1360 (e.g., around the perimeter of the well region). As a result, samples in sample plate 1350 heated using adaptor plate 1300 will suffer from thermal edge effects. This is because conventional adaptor plates heat the sample plates primarily via conductive heat transfer. In contrast, adaptor plates in accordance with embodiments of the present disclosure use a combination of conductive and convective heating to achieve a uniform temperature distribution across the wells. For instance, because the chambers provide convective and conductive heating (e.g., instead of primarily conductive heating) the outer-wells (e.g., outer well 2520 in FIG. 2D) will not rise in temperature at a similar rate to the rise in temperature of the inner-wells (e.g., inner well 252i in FIG. 2D).

Accordingly, embodiments of the present disclosure provide a DNA extractor system comprising an adaptor plate 400, a sample plate 450, and a heater 180 that can achieve a uniform temperature distribution (e.g., where the samples have a temperature deviation of about 2 C) of the sample material contained in the plurality of wells of the sample plate. Specific exemplary methods of using a DNA extractor system in accordance with embodiments of this disclosure are discussed in greater detail below.

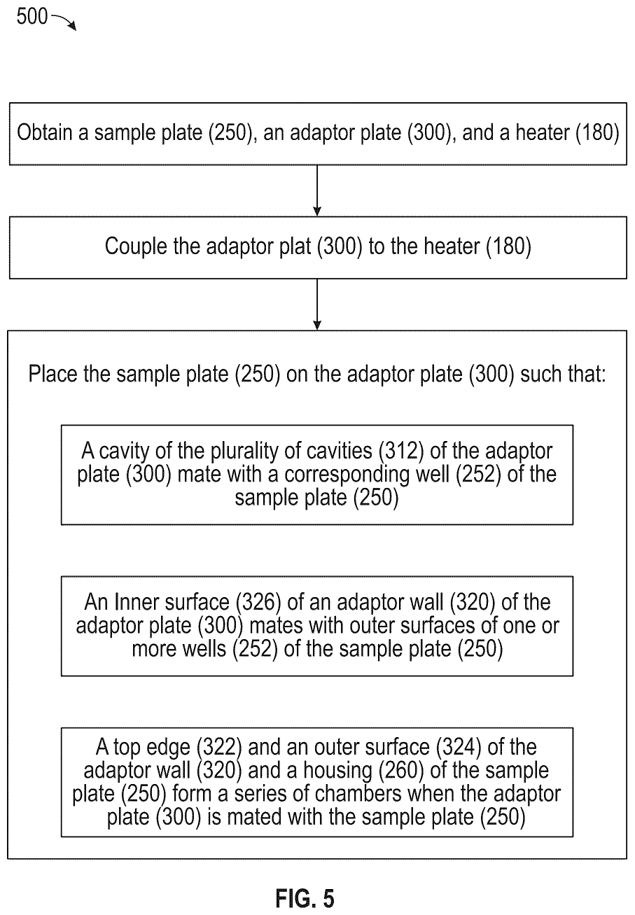

FIG. 5 illustrates an exemplary process 500 for installing a sample plate (e.g., sample plate 140, 250, 450) and an adaptor plate (e.g., adaptor plate 100, 300, 400) to a heater (e.g., heater 180), in accordance with some embodiments. While reference numerals are provided with respect to specific figures, it is to be understood that the following process may be performed on corresponding components described herein. In the process 500, the adaptor plate may comprise a receptacle region 310, the receptacle region comprising a plurality of cavities 312; an adaptor wall 320 disposed along a perimeter of the receptacle region 310, the adaptor wall 320 comprising: an inner surface 326; a top edge 322, and an outer surface 324 connected to the inner surface 326 by the top edge 322. In some embodiments, the sample plate used in process 500 may comprise a housing, the housing 260 having an upper surface 266 and an outer wall 262. The sample plate 250 may further comprise a well-region, the well-region comprising a plurality of wells 252 disposed within the housing 260. In some embodiments, a well 252 of the plurality of wells 252 comprise an opening located at the upper surface 266 of the housing 260, a sidewall 254 extending from the opening into the housing 260, a bottom well-surface 258, the bottom well-surface 258 configured to mate with the cavity 312 of the adaptor plate 300, and a plurality of ribs 264, the plurality of ribs 264 disposed within the housing 260 and extending from the sample-well region to the outer wall 262. In some embodiments, the sample plate may be configured to contain one or more biological samples (e.g., tumor samples, tissue samples, blood samples, etc.).

The process 500 may be performed manually (e.g., by a technician) and/or automatically (e.g., by a robotic device). At block 502, the sample plate 250, the adaptor plate 300, and the heater 180 may be obtained. In some embodiments, obtaining the adaptor plate 300 may comprise forming the adaptor plate 300 via one or more of casting, stamping or forging, additive manufacturing (e.g., fused-deposition modeling, selective-layer-sintering, etc.), subtractive manufacturing (e.g., CNC milling, electrical-discharge machining, etc.), or a combination thereof.

At block 504, the adaptor plate 300 can be coupled to the heater 180. In some embodiments, coupling the adaptor plate 300 to the heater can comprise fastening the adaptor plate 300 to the heater 180. For instance, one or more fasteners, e.g., one or more screws or bolts, is used to secure the adaptor plate 300 to the heater 180. For example, in some embodiments a fastener may be inserted into an aperture 328 of the adaptor plate 300 and further inserted into a corresponding aperture on the heater 180. In some embodiments the adaptor plate 300 may be secured to the heater 180 using an adhesive. In this manner, the adaptor plate can be secured to the heater 180 to remain mounted to the heater during vibratory motion of the heater. In some embodiments, prior to coupling the adaptor plate to the heater, a thermal paste and/or thermal transfer fluid maybe be applied to either the heater 180 or the adaptor plate 300, such that the thermal paste and/or thermal transfer fluid is disposed between the adaptor plate 300 and the heater 180. The thermal paste and/or thermal transfer fluid may improve conduction of heat between the adaptor plate 300 and the heater 180.

At block 506, the sample plate 250 can be placed on the adaptor plate 300. In some embodiments, the sample plate 250 can be placed on the adaptor plate 300 such that a cavity of the plurality of cavities 312 of the adaptor plate 300 mates with a corresponding well 252 of the sample plate 250; an inner surface 326 of the adaptor wall 320 mates with outer surfaces of one or more wells 252 of the sample plate 250; and a top edge 322 and the outer surface 324 of the adaptor wall 320 and a housing 260 of the sample plate 250 form a series of chambers when the adaptor plate 300 is mated with the sample plate 250.

For instance, when the sample plate 250 is placed on the adaptor plate 300 a bottom surface of the sample plate 250 can mate with an upper surface of the plurality of cavities 312 of the receptacle region of the adaptor plate 300. Additionally, the inner surface 326 of the adaptor wall 320 can mate with outer surfaces of one or more wells 252 of the sample plate 250. In this manner, the adaptor plate 300 can conduct heat to the bottom surface of the each of the wells as well as the outer wells (e.g., wells along the perimeter) of the sample plate 250 via conduction. In some embodiments, prior to placing the sample plate 250 on the adaptor, a thermal paste and/or thermal transfer fluid maybe be applied to either the sample plate 250 or the adaptor plate 300, such that the thermal paste and/or thermal transfer fluid is disposed between the adaptor plate 300 and the sample plate 250. The thermal paste and/or thermal transfer fluid may improve conduction of heat between the adaptor plate 300 and the sample plate 250.

In some embodiments, the sample plate 250 can be placed on the adaptor plate 300 such that a top edge 322 and the outer surface 324 of the adaptor wall 320 and a housing 260 of the sample plate 250 form a series of chambers when the adaptor plate 300 is mated with the sample plate 250. In some embodiments, the series of chamber may comprise a fluid (e.g., air) such that heat from the fluid disposed in the chambers heats the sample plate 250 via convection. In one or more examples, the adaptor plate 300 is configured to form a first gap between the top edge 322 of the adaptor wall 320 and the housing 260 of the sample plate 250 when the adaptor plate 300 is mated with the sample plate 250 and/or placed on the adaptor plate. The first gap may correspond to a height differential between the housing 260 of the sample plate 250 and the top edge 322. In one or more examples, the adaptor plate 300 is configured to form a second gap between the outer surface 324 of the adaptor wall 320 and an outer wall 262 of the housing 260 of the sample plate 250 when the adaptor plate 300 is mated with the sample plate 250 and/or the sample plate is placed on the adaptor plate, the second gap different than the first gap. The first gap and the second may define one or more dimensions of the series of chambers.

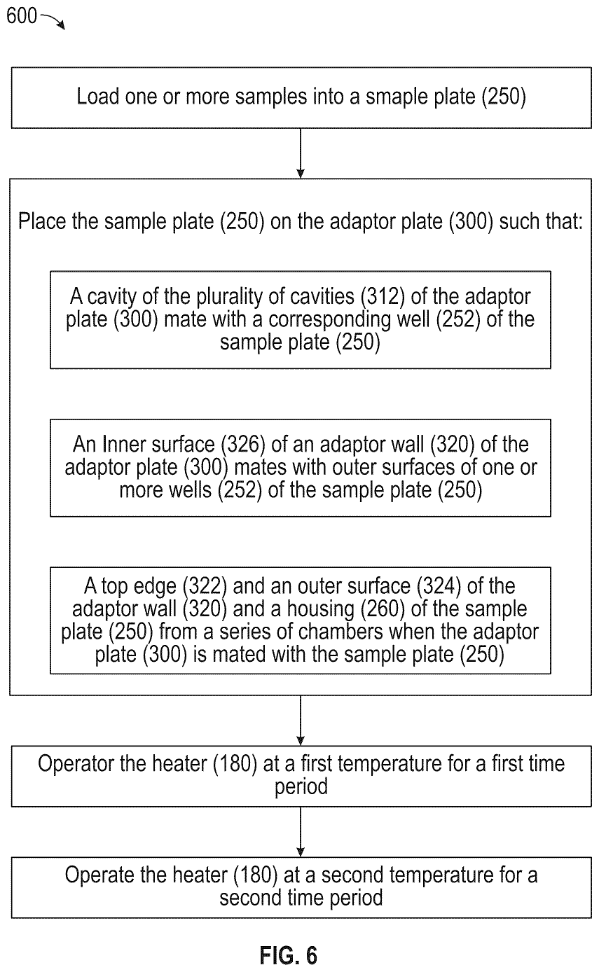

FIG. 6 illustrates an exemplary process 600 for heating one or more samples disposed in a sample plate (e.g., sample plate 150, 250, 450) according to embodiments of this disclosure. A DNA extractor system comprising a sample plate, an adaptor plate (e.g., adaptor plate 100, 300, 400), and a heater (e.g., heater 180) may be used to perform process 600. The process 600 may be performed manually (e.g., by a technician) and/or automatically (e.g., by a robotic device). While reference numerals are provided with respect to specific figures, it is to be understood that the following process may be performed using any of the embodiments disclosed herein.

In process 600, the adaptor plate 300 may comprise a receptacle region 310, the receptacle region comprising a plurality of cavities 312; an adaptor wall 320 disposed along a perimeter of the receptacle region 310, the adaptor wall 320 comprising: an inner surface 326; a top edge 322, and an outer surface 324 connected to the inner surface 326 by the top edge 322.

The process 600 may further use a sample plate, e.g., sample plate 250. In some embodiments the sample plate may comprise a housing, the housing 260 having an upper surface 266 and an outer wall 262. The sample plate 250 may further comprise a well-region, the well-region comprising a plurality of wells 252 disposed within the housing 260. In some embodiments, a well 252 of the plurality of wells 252 comprise an opening located at the upper surface 266 of the housing 260, a sidewall 254 extending from the opening into the housing 260, a bottom well-surface 258, the bottom well-surface 258 configured to mate with the cavity 312 of the adaptor plate 300, and a plurality of ribs 264, the plurality of ribs 264 disposed within the housing 260 and extending from the sample-well region to the outer wall 262. In some embodiments, the sample plate may be configured to contain one or more biological samples (e.g., tumor samples, tissue samples, blood samples, etc.).

The process 600 may further use a heater, e.g., heater 180. In one or more examples the heater may be configured to have a heating set point up to 99 C with an accuracy of 0.1 C and a hot plate uniformity of 0.5 C. In one or more examples, the heater may be capable of mechanically agitating the samples in the sample place and have a 3 mm orbital mixing frequency of 200 to 2000 rpm and a mechanical sizing to accommodate large-volume sample plates. It will be appreciated that heaters that can accommodate large-volume sample plates may be used in accordance with embodiments of this disclosure.

At block 602, one or more samples can be loaded into a sample plate 250. For instance, a volume of about 7-10 mL may be provided to one or more wells of the sample plate 250. In some embodiments, the sample may have previously been obtained from a patient. In some embodiments, the one or more samples may comprise at least one DNA molecule.

At block 604, the sample plate 250 can be placed on the adaptor plate 300. That is, the sample plate loaded with one or more samples can be placed on the adaptor plate. In one or more embodiments, the adaptor plate may already be mounted to the heater 180 prior to block 604. In some embodiments, the sample plate 250 can be placed on the adaptor plate 300 such that a cavity of the plurality of cavities 312 of the adaptor plate 300 mates with a corresponding well 252 of the sample plate 250; an inner surface 326 of the adaptor wall 320 mates with outer surfaces of one or more wells 252 of the sample plate 250; and a top edge 322 and the outer surface 324 of the adaptor wall 320 and a housing 260 of the sample plate 250 form a series of chambers when the adaptor plate 300 is mated with the sample plate 250.

For instance, when the sample plate 250 is placed on the adaptor plate 300 a bottom surface of the sample plate 250 can mate with an upper surface of the plurality of cavities 312 of the receptacle region of the adaptor plate 300. Additionally, the inner surface 326 of the adaptor wall 320 can mate with outer surfaces of one or more wells 252 of the sample plate 250. In this manner, the adaptor plate 300 can conduct heat to the bottom surface of the each of the wells as well as the outer wells (e.g., wells along the perimeter) of the sample plate 250 via conduction. In some embodiments, prior to placing the sample plate 250 on the adaptor, a thermal paste and/or thermal transfer fluid maybe be applied to either the sample plate 250 or the adaptor plate 300, such that the thermal paste and/or thermal transfer fluid is disposed between the adaptor plate 300 and the sample plate 250. The thermal paste and/or thermal transfer fluid may improve conduction of heat between the adaptor plate 300 and the sample plate 250.

In some embodiments, the sample plate 250 can be placed on the adaptor plate 300 such that a top edge 322 and the outer surface 324 of the adaptor wall 320 and a housing 260 of the sample plate 250 form a series of chambers when the adaptor plate 300 is mated with the sample plate 250. In some embodiments, the series of chamber may comprise a fluid (e.g., air) such that heat from the fluid disposed in the chambers heats the sample plate 250 via convection. In one or more examples, the adaptor plate 300 is configured to form a first gap between the top edge 322 of the adaptor wall 320 and the housing 260 of the sample plate 250 when the adaptor plate 300 is mated with the sample plate 250 and/or placed on the adaptor plate. The first gap may correspond to a height differential between the housing 260 of the sample plate 250 and the top edge 322. In one or more examples, the adaptor plate 300 is configured to form a second gap between the outer surface 324 of the adaptor wall 320 and an outer wall 262 of the housing 260 of the sample plate 250 when the adaptor plate 300 is mated with the sample plate 250 and/or the sample plate is placed on the adaptor plate, the second gap different than the first gap. The first gap and the second may define one or more dimensions of the series of chambers.

At step 606, the heater can be operated at a first temperature for a first time period. As discussed above, the target temperature for DNA extraction is 60 C. The heater can be operated at the first temperature to rapidly increase the temperature of the one or more samples disposed in the sample plate 250 to approach the target temperature. In some embodiments, the first temperature may correspond to a temperature above the target temperature of 60 C. In some embodiments, the heater 180 can be operated at temperature of about 90-99 C for the first time period. In some embodiments, the heater can be operated at a temperature of about 95 C for the first time period.

The first time period may be selected to correspond to an amount of time it takes for one or more samples disposed in the sample plate 250 to be within 5-10 C of the target temperature of 60 C when heated at the first temperature. In some embodiments, the first time period may be based on the first temperature. For instance, a lower first temperature may be associated with a longer first time period. In one or more examples, the first time period may correspond to 10-20 minutes. In some embodiments, the first time period may correspond to 15 minutes when the first temperature corresponds to 95 C. In one or more examples, the heater may further be operated to mechanically agitate, e.g., shake, the one or more samples in the sample plate, at a first speed. In some embodiments, the first speed may correspond to about 500-800 rpm. In one or more examples, the speed may be set as high as possible while avoiding significant sample contact with the seal at the top of the well chimney, e.g., at the opening of the well which helps reduce risk of cross-contamination between samples. Agitating or shaking the sample mixture during digestion allows heat to rapidly distribute from the bottom and/or side of the well chambers to achieve a uniform mixture of the sample and the extraction reagents.

At step 608, the heater can be operated at a second temperature for a second time period. The heater may be operated at the second temperature to gradually increase the temperature of the one or more samples of the sample plate once the sample temperature is within 5-10 C of the target temperature of 60 C. In this manner, the DNA extractor system ensures that the one or more samples are not overheated, which could damage the sample and adversely impact the DNA yields.

Accordingly, embodiments of the present disclosure provide a DNA extractor system comprising an adaptor plate 400, a sample plate 450, and a heater 180 that can achieve a uniform temperature distribution (e.g., where the samples have a temperature deviation of about 2 C) of the sample material contained in the plurality of wells of the sample plate. For instance, this uniform temperature distribution of the sample wells can be achieved due to the combination of conductive and convective heating between the sample plate and the adaptor plate. Specifically, the adaptor plate was designed to minimize thermal edge effects, whereby outer wells (e.g., wells disposed on a perimeter of the sample plate) heat more quickly than inner wells of the sample plate.

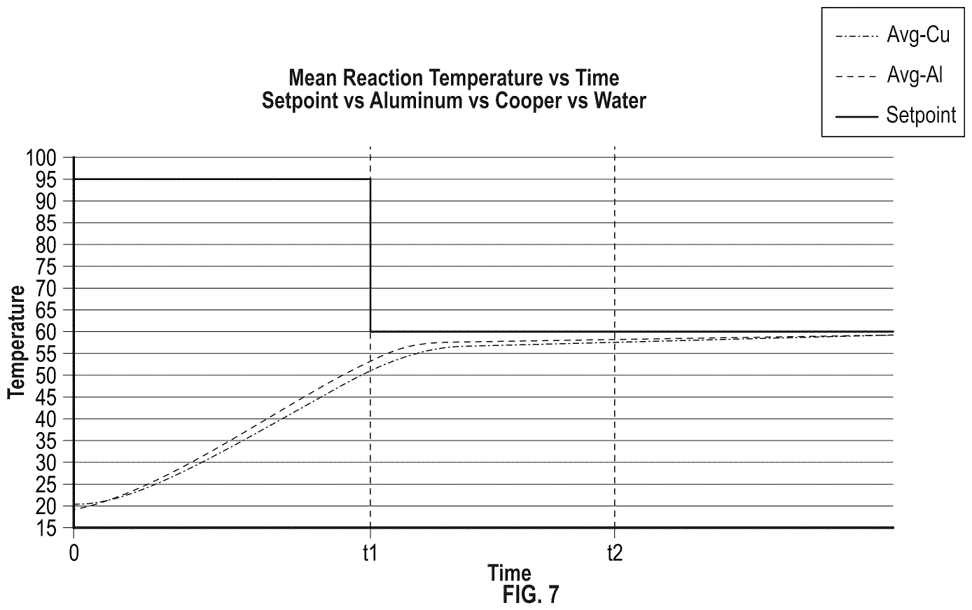

FIG. 7 illustrates an exemplary plot that shows the average reaction temperature over time for the heater, a sample disposed in a sample plate formed from aluminum, a sample disposed in a sample plate formed form copper. As shown in the figure, the at time to, the heater may be operated at 95 C, while the copper and aluminum sample plates are at an ambient temperature (e.g., around 20 C). During the first time period between t0 and t1, the copper and aluminum sample plates may quickly increase in temperature from the ambient conditions to a predetermined temperature, where the predetermined temperature is about 5-10 C less than a target temperature. At time t1, the temperature of the heater may be decreased from the first temperature to the target temperature (e.g., the temperature of the heater may be decreased from 95 C to 60 C). After time t1, the temperature of the samples in the copper and aluminum sample plates may more gradually approach the target temperature of 60 C. At time t2, the samples in the copper and aluminum sample plates may have reached a steady state near 60 C (e.g., within 0-2 C of the target temperature).

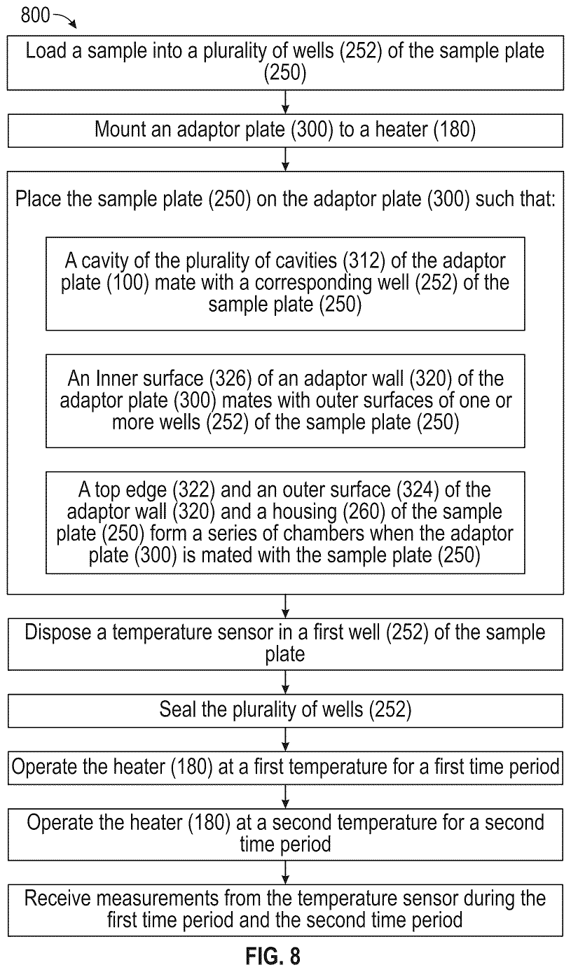

FIG. 8 illustrates an exemplary process 800 for heating one or more samples disposed in a sample plate (e.g., sample plate 150, 250, 450) according to embodiments of this disclosure. A DNA extractor system comprising a sample plate, an adaptor plate (e.g., adaptor plate 100, 300, 400), and a heater (e.g., heater 180) may be used to perform process 800. The process 800 may be performed manually (e.g., by a technician) and/or automatically (e.g., by a robotic device). While reference numerals are provided with respect to specific figures, it is to be understood that the following process may be performed using any of the embodiments disclosed herein.

In process 800, the adaptor plate 300 may comprise a receptacle region 310, the receptacle region comprising a plurality of cavities 312; an adaptor wall 320 disposed along a perimeter of the receptacle region 310, the adaptor wall 320 comprising: an inner surface 326; a top edge 322, and an outer surface 324 connected to the inner surface 326 by the top edge 322.

The process 800 may further use a sample plate, e.g., sample plate 250. In some embodiments the sample plate may comprise a housing, the housing 260 having an upper surface 266 and an outer wall 262. The sample plate 250 may further comprise a well-region, the well-region comprising a plurality of wells 252 disposed within the housing 260. In some embodiments, a well 252 of the plurality of wells 252 comprise an opening located at the upper surface 266 of the housing 260, a sidewall 254 extending from the opening into the housing 260, a bottom well-surface 258, the bottom well-surface 258 configured to mate with the cavity 312 of the adaptor plate 300, and a plurality of ribs 264, the plurality of ribs 264 disposed within the housing 260 and extending from the sample-well region to the outer wall 262. In some embodiments, the sample plate may be configured to contain one or more biological samples (e.g., tumor samples, tissue samples, blood samples, etc.).

The process 800 may further use a heater, e.g., heater 180. In some embodiments, the heater 180 may be a bioshaker or thermoshaker. In one or more examples the heater may be configured to have a heating set point up to 99 C with an accuracy of 0.1 C and a hot plate uniformity of 0.5 C. In one or more examples, the heater may be capable of mechanically agitating the samples in the sample place and have a 3 mm orbital mixing frequency of 200 to 2000 rpm and a mechanical sizing to accommodate large-volume sample plates. It will be appreciated that heaters that can accommodate large-volume sample plates may be used in accordance with embodiments of this disclosure.

At block 802, one or more samples can be loaded into a sample plate 250. For instance, volume of about 7-10 mL may be provided to one or more wells 252 of the sample plate 250. In some embodiments, the sample may have previously been obtained from a patient. In some embodiments, the one or more samples may comprise at least one DNA molecule.

At block 804, the adaptor plate 300 can be mounted to a heater 180. In some embodiments, the adaptor plate 300 can be mounted to the heater using one or more fasteners, e.g., one or more screws or bolts. In some embodiments a fastener may be inserted into an aperture 328 of the adaptor plate 300 and further inserted into a corresponding aperture on the heater 180. In some embodiments, the adaptor plate 300 can be secured to the heater 180 with the fastener. In this manner, the adaptor plate can be secured to the heater 180 for any vibratory motion of the heater. In some embodiments, prior to coupling the adaptor plate to the heater, a thermal paste and/or thermal transfer fluid may be applied to either the heater 180 or the adaptor plate 300, such that the thermal paste and/or thermal transfer fluid is disposed between the adaptor plate 300 and the heater 180. The thermal paste and/or thermal transfer fluid may improve conduction of heat between the adaptor plate 300 and the heater 180.

At block 806, the sample plate 250 can be placed on the adaptor plate 300. That is, the sample plate loaded with one or more samples can be placed on the adaptor plate. In one or more embodiments, the adaptor plate may already be mounted to the heater 180 prior to block 806. In some embodiments, the sample plate 250 can be placed on the adaptor plate 300 such that a cavity of the plurality of cavities 312 of the adaptor plate 300 mates with a corresponding well 252 of the sample plate 250; an inner surface 326 of the adaptor wall 320 mates with outer surfaces of one or more wells 252 of the sample plate 250; and a top edge 322 and the outer surface 324 of the adaptor wall 320 and a housing 260 of the sample plate 250 form a series of chambers when the adaptor plate 300 is mated with the sample plate 250.

For instance, when the sample plate 250 is placed on the adaptor plate 300 a bottom surface of the sample plate 250 can mate with an upper surface of the plurality of cavities 312 of the receptacle region of the adaptor plate 300. Additionally, the inner surface 326 of the adaptor wall 320 can mate with outer surfaces of one or more wells 252 of the sample plate 250. In this manner, the adaptor plate 300 can conduct heat to the bottom surface of the each of the wells as well as the outer wells (e.g., wells along the perimeter) of the sample plate 250 via conduction. In some embodiments, prior to placing the sample plate 250 on the adaptor, a thermal paste and/or thermal transfer fluid maybe be applied to either the sample plate 250 or the adaptor plate 300, such that the thermal paste and/or thermal transfer fluid is disposed between the adaptor plate 300 and the sample plate 250. The thermal paste and/or thermal transfer fluid may improve conduction of heat between the adaptor plate 300 and the sample plate 250.

In some embodiments, the sample plate 250 can be placed on the adaptor plate 300 such that a top edge 322 and the outer surface 324 of the adaptor wall 320 and a housing 260 of the sample plate 250 form a series of chambers when the adaptor plate 300 is mated with the sample plate 250. In some embodiments, the series of chamber may comprise a fluid (e.g., air) such that heat from the fluid disposed in the chambers heats the sample plate 250 via convection. In one or more examples, the adaptor plate 300 is configured to form a first gap between the top edge 322 of the adaptor wall 320 and the housing 260 of the sample plate 250 when the adaptor plate 300 is mated with the sample plate 250 and/or placed on the adaptor plate. The first gap may correspond to a height differential between the housing 260 of the sample plate 250 and the top edge 322. In one or more examples, the adaptor plate 300 is configured to form a second gap between the outer surface 324 of the adaptor wall 320 and an outer wall 262 of the housing 260 of the sample plate 250 when the adaptor plate 300 is mated with the sample plate 250 and/or the sample plate is placed on the adaptor plate, the second gap different than the first gap. The first gap and the second may define one or more dimensions of the series of chambers.

At block 808, a temperature sensor can be disposed in a first well 252 of the sample plate. In one or more examples, the temperature sensor can comprise one or more sensor leads that can be placed in the sample disposed in the first well. In some embodiments, multiple leads of one or more temperature sensors may be placed in a corresponding well. For example, a second lead of a second temperature sensor may be disposed in a second well of the sample plate. In this manner, the one or more temperature sensors can output one or more signals indicative of the temperature of the sample disposed in a corresponding well.

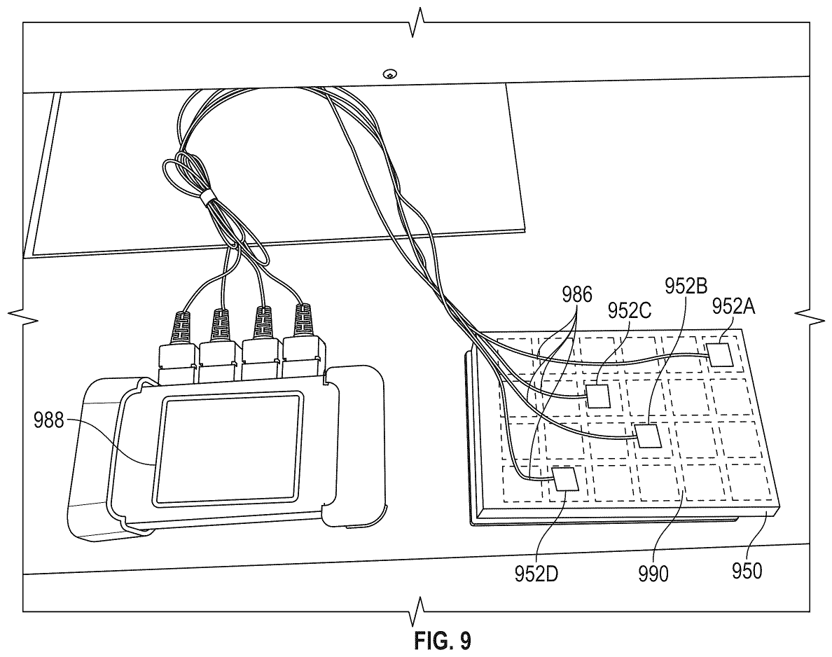

FIG. 9 illustrates an exemplary set-up for measuring the temperature of samples disposed in one or more wells of a sample plate. The exemplary set-up can include a sample plate 950 with a plurality of samples, an adaptor plate (not shown), a heater (not shown), one or more temperature sensors 986, and one or more devices 488 coupled to the temperature sensor. As shown in the figure, lead lines of a temperature sensors 486 are disposed in corresponding wells 952A-952D of sample plate 950. Wells 952A-952D were selected to provide a representative temperature distribution of the wells included in the sample plate 950. For instance, well 952A is disposed in a corner of the well region of sample plate 950, and thus is more impacted by thermal edge effects because two of the sidewalls of the well 952A are configured to mate with the adaptor plate (e.g., adaptor plate 300). Well 952D is disposed along a perimeter of the well region of the sample plate 950, such that one sidewall mates with the adaptor plate 300. Wells 952B and 952C are disposed within an interior portion of the well region, such that none of the sidewall of these wells mate with the adaptor plate.

At block 810, the plurality of wells 252 may be sealed. In some embodiments, sealing the plurality of wells 252 may ensure that the temperature leads are secured in a corresponding well. In some embodiments, sealing the plurality of wells may reduce heat loss of the sample via an opening of the well. In some embodiments, sealing the plurality of wells may prevent spillage of the sample during vibratory motion of the heater. Referring again to FIG. 9, the seal may be placed over the upper surface (e.g., upper surface 266) and the openings of the plurality of wells.

At block 812, the heater can be operated at a first temperature for a first period of time. In one or more embodiments, block 812 can correspond to block 606 of process 600. For example, the heater can be operated at the first temperature to rapidly increase the temperature of the one or more samples disposed in the sample plate 250. In some embodiments, the first temperature may correspond to a temperature above the target temperature of 60 C. In some embodiments, the first time period is selected to correspond to an amount of time it takes for one or more samples disposed in the sample plate 250 to be within 5-10 C of the target temperature of 60 C when heated at the first temperature. In one or more examples, the heater may further be operated to mechanically agitate, e.g., shake, the one or more samples in the sample plate, at a first speed.

At block 814, the heater can be operated at a second temperature for a second time period. In one or more examples, block 814 can correspond to block 608 of process 600. For instance, the heater may be operated at the second temperature to gradually increase the temperature of the one or more samples of the sample plate once the sample temperature is within 5-10 C of the target temperature of 60 C. In this manner, the DNA extractor system ensures that the one or more samples are not overheated, which could damage the sample and adversely impact the DNA yields.

At block 816, the DNA extractor can receive one or more measurements from the temperature sensor during the first time period and the second time period. For instance, the data included in the plot 700 may correspond to one or more measurements received from a temperature sensor during the first time period and the second time period.

In some embodiments, the temperature measurements from the one or more wells can be used to refine the design of an adaptor plate in accordance with embodiments of the present disclosure. For instance, if the temperature measurements indicate that the measured samples are impacted by thermal edge effects, one or more dimensions of the adaptor plate can be changed to improve the uniformity of the temperature distribution. For instance, if the samples appear to be impacted by thermal edge effects, a thickness and/or height of the adaptor wall may be reduced.

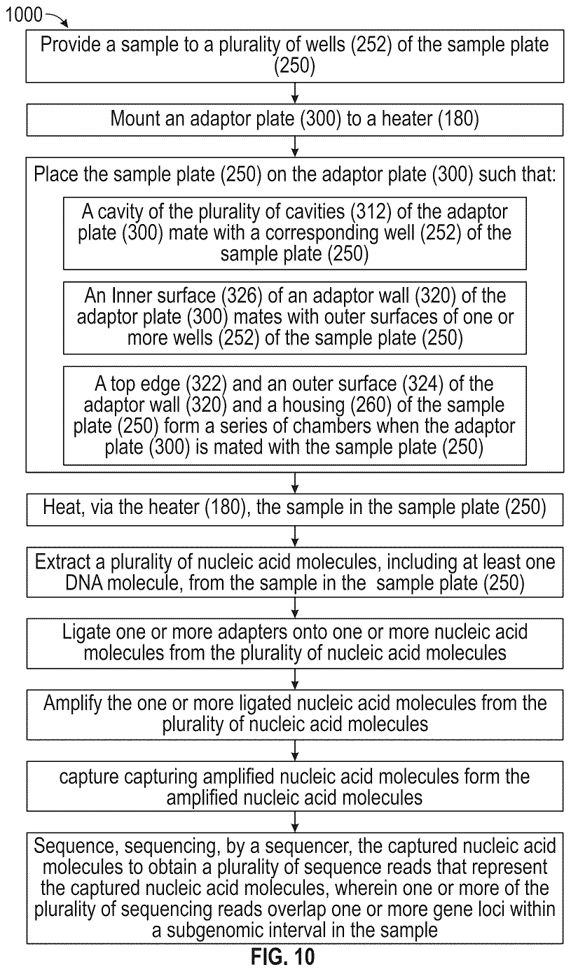

FIG. 10 illustrates an exemplary process 1000 for heating, via an adaptor plate, one or more samples disposed in a sample plate and then sequencing a sample of nucleic acid molecules, in accordance with some embodiments. A DNA extractor system comprising a sample plate, an adaptor plate (e.g., adaptor plate 100, 300, 400), and a heater (e.g., heater 180) may be used to perform process 1000. The process 1000 may be performed manually (e.g., by a technician) and/or automatically (e.g., by a robotic device). While reference numerals are provided with respect to specific figures, it is to be understood that the following process may be performed using any of the embodiments disclosed herein.

In process 1000, the adaptor plate 300 may comprise a receptacle region 310, the receptacle region comprising a plurality of cavities 312; an adaptor wall 320 disposed along a perimeter of the receptacle region 310, the adaptor wall 320 comprising: an inner surface 326; a top edge 322, and an outer surface 324 connected to the inner surface 326 by the top edge 322.

The process 1000 may further use a sample plate, e.g., sample plate 250. In some embodiments the sample plate may comprise a housing, the housing 260 having an upper surface 266 and an outer wall 262. The sample plate 250 may further comprise a well-region, the well-region comprising a plurality of wells 252 disposed within the housing 260. In some embodiments, a well 252 of the plurality of wells 252 comprise an opening located at the upper surface 266 of the housing 260, a sidewall 254 extending from the opening into the housing 260, a bottom well-surface 258, the bottom well-surface 258 configured to mate with the cavity 312 of the adaptor plate 300, and a plurality of ribs 264, the plurality of ribs 264 disposed within the housing 260 and extending from the sample-well region to the outer wall 262. In some embodiments, the sample plate may be configured to contain one or more biological samples (e.g., tumor samples, tissue samples, blood samples, etc.).

The process 1000 may further use a heater, e.g., heater 180. In some embodiments, the heater 180 may be a bioshaker or thermoshaker. In one or more examples the heater may be configured to have a heating set point up to 99 C with an accuracy of 0.1 C and a hot plate uniformity of 0.5 C. In one or more examples, the heater may be capable of mechanically agitating the samples in the sample place and have a 3 mm orbital mixing frequency of 200 to 2000 rpm and a mechanical sizing to accommodate large-volume sample plates. It will be appreciated that heaters that can accommodate large-volume sample plates may be used in accordance with embodiments of this disclosure.

At block 1002, one or more samples can be provided to a plurality of wells 252 of a sample plate 250. In one or more embodiments, block 1002 can correspond to block 802 of process 800. At block 1004, the adaptor plate 300 can be mounted to the heater. In one or more embodiments, block 1004 can correspond to block 804 of process 800. At block 1006, the sample plate 250 can be placed on the adaptor plate 300. In one or more embodiments, block 1006 can correspond to block 806 of process 800. At block 1008, the one or more samples in the sample plate 250 can be heated using heater 180. In one or more embodiments, block 1008 can correspond to blocks 812 and 814 of process 800.

At block 1010, a plurality of nucleic acid molecules may be extracted from the sample contained in the sample plate. In some embodiments, the plurality of nucleic acid molecules obtained from the sample may comprise at least one DNA molecule. At block 1012, one or more adapters may be ligated onto one or more nucleic acid molecules from the plurality of nucleic acid molecules. At block 1014, one or more of the ligated nucleic acid molecules from the plurality of nucleic acid molecules may be amplified. At block 1016, amplified nucleic acid molecules may be captured. Finally, at block 1018, the captured nucleic acid molecules may be sequenced by a sequencer in order to obtain a plurality of sequence reads that represent the captured nucleic acid molecules. In some embodiments of the process 1000, one or more of the plurality of sequencing reads may overlap one or more gene loci within a sub-genomic interval in the sample.

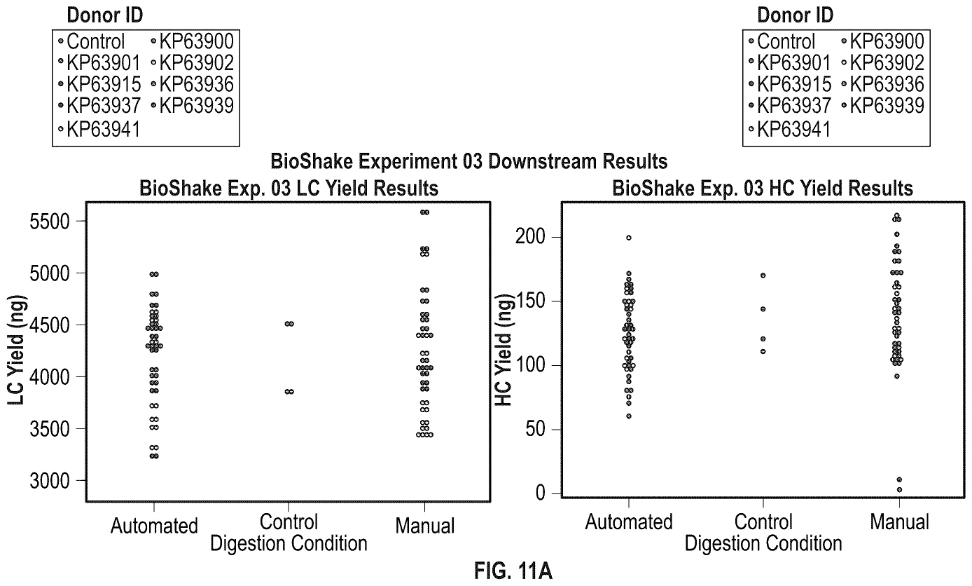

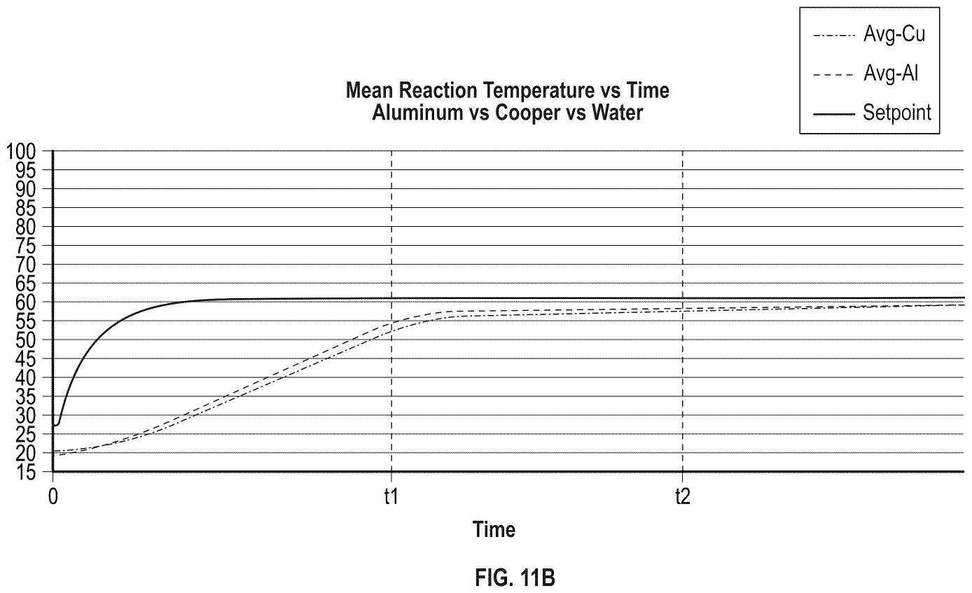

FIG. 11A is an exemplary plot that illustrates a comparison of the downstream DNA extraction results using methods for heating a sample in accordance with embodiments of this disclosure and manual methods for heating a sample. As shown in the plot, both methods output good yields within the desired range. Additionally, the plots indicate substantial overlap in yield concentrations when performing the manual methods and methods in accordance with embodiments of this disclosure, on replicate (e.g., duplicated) samples.

The manual methods to heat the sample comprise placing individual vials of a 7-10 mL sample in a water bath with a set point of 60 C. FIG. 11B illustrates an exemplary temperature profile that compares the for two vials that are disposed in the water bath. As shown in the figure, the water bath samples rapidly reach a steady state temperature before the samples disposed in the aluminum and copper adaptor plates. However, the water bath method suffers from being very time and labor intensive. Accordingly, embodiments of the present disclosure provide an efficient and method for rapidly heating multiple samples for DNA extraction using a DNA extractor system and adaptor plate in accordance with embodiments of the present disclosure.

The disclosed systems and methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods and systems may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods and systems may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods and systems may be used in treating a disease (e.g., a cancer) in a subject. For example, in response to analyzing the samples in the sample plate using any of the methods disclosed herein, an effective amount of an anti-cancer therapy or anti-cancer treatment may be administered to the subject.

In some instances, the methods and systems can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde-or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D 3399-00, D 3399-01, and D 3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A. ® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may omprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000x or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Systems

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for analyzing any of a variety of samples as described herein (e.g., a tissue sample, biopsy sample, hematological sample, or liquid biopsy sample derived from the subject).

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EXEMPLARY IMPLEMENTATIONS

Exemplary implementations of the methods and systems described herein include:

1. An adaptor plate (300) configured to couple a sample plate (250) to a heater (180), the adaptor plate (300) comprising:
a receptacle region (310), the receptacle region comprising a plurality of cavities, at least one cavity (312) of the plurality of cavities configured to mate with a corresponding well (252) of the sample plate (250);
an adaptor wall (320) disposed along a perimeter of the receptacle region (310), the adaptor wall (320) comprising:
   an inner surface (326) configured to contact outer surfaces of one or more wells (252) of the sample plate (250);
   an outer surface (324); and
   a top edge (322) connecting the outer surface (324) to the inner surface (326), wherein the top edge (322) and the outer surface (324) of the adaptor wall and a housing (260) of the sample plate (250) are configured to form a series of chambers when the adaptor plate (300) is mated with the sample plate (250); and a bottom surface (306) configured to mate with the heater (180).
      2. The adaptor plate of clause 1, wherein the adaptor plate (300) is configured to form a first gap (382) between the top edge (322) of the adaptor wall (320) and the housing (260) of the sample plate (250) when the adaptor plate (300) is mated with the sample plate (250).
      3. The adaptor plate of clause 1 or clause 2, wherein the adaptor plate (300) is configured to form a second gap (384) between the outer surface (324) of the adaptor wall (320) and an outer wall (262) of the housing (260) of the sample plate (250) when the adaptor plate (300) is mated with the sample plate (250), the second gap different than the first gap.
      4. The adaptor plate of clauses 1-3, wherein the adaptor wall (320) is configured to transfer heat from the heater (180) to the sample plate (250) via conduction.
      5. The adaptor plate of clauses 1-4, wherein, fluid disposed in the series of chambers is configured to transfer heat from the heater (180) to the sample plate (250) via convection.
      6. The adaptor plate of clauses 1-5, wherein the adaptor plate (300) is configured to mate with a second sample plate, the second sample plate different from the first sample plate.
      7. The adaptor plate of clauses 1-6, wherein the adaptor wall (320) comprises a plurality of slots (314), a slot of the plurality of slots configured to receive a rib (264) of the sample plate (250).
      8 The adaptor plate of clauses 1-7, wherein the plurality of cavities (312) of the receptacle region (310) are arranged in a grid.
      9. The adaptor plate of clauses 1-8, wherein a shape of the at least one cavity (312) comprises an inverted pyramid, a hemi-sphere, an inverted cone, or an inverted top-hat profile.
      10. The adaptor plate of clauses 1-9, wherein the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.
      11. The adaptor plate of clauses 1-10, the adaptor plate (300) is formed by casting, stamping or forging, additive manufacturing, subtractive manufacturing or a combination thereof.
      12. The adaptor plate of clauses 1-11, the adaptor plate further comprising: a flange (304) disposed along a foot of the outer surface (324) of the adaptor wall (320), the flange configured to form the series of chambers.
      13. A system comprising:
a sample plate (250);
an adaptor plate (300) configured to mate with the sample plate (250), the adaptor plate (300) comprising:
   a receptacle region (310), the receptacle region comprising a plurality of cavities, at least one cavity (312) of the plurality of cavities configured to mate with at least one corresponding well (252) of the sample plate (250);
   an adaptor wall (320) disposed along a perimeter of the receptacle region (310), the adaptor wall (320) comprising:
      an inner surface (326) configured to contact outer surfaces of one or more wells (252) of the sample plate (250);
      an outer surface (324); and
      a top edge (322) connecting the outer surface (324) to the inner surface (326), wherein the top edge (322) and the outer surface (324) of the adaptor wall (320) and a housing (260) of the sample plate (250) are configured to form a series of chambers (370) when the adaptor plate (300) is mated with the sample plate (250); and
      a bottom surface (306); and
a heater (180) configured to mate with the bottom surface (306) of the adaptor plate (300).
   14. The system of clause 13, wherein the sample plate (250) comprises:
the housing (260) having an upper surface (266) and an outer wall (262);
a well-region, the well-region comprising a plurality of wells (252) disposed within the housing (260), wherein the at least one corresponding well (252) of the plurality of wells (252) comprises:
   an opening located at the upper surface (266) of the housing (260);
   a sidewall (254) extending from the opening into the housing (260);
   a bottom well-surface (258), the bottom well-surface (258) configured to mate with the at least cavity (312) of the adaptor plate (300); and
a plurality of ribs (264), the plurality of ribs (264) disposed within the housing (260) and extending from the sample-well region to the outer wall (262).
   15. The system of clause 13 or clause 14, wherein the heater comprises a thermoshaker.
   16. The system of clauses 13-15, wherein the adaptor plate (300) is configured to form a first gap between the top edge (322) of the adaptor wall (320) and the housing (260) of the sample plate (250) when the adaptor plate (300) is mated with the sample plate (250).
   17. The system of clauses 13-16, wherein the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap.
   18. The system of clauses 13-17, wherein, the adaptor wall (320) is configured to transfer heat from the heater (180) to the sample plate (250) via conduction.
   19. The system of clauses 13-18, wherein, fluid disposed in the series of chambers is configured to transfer heat from the heater (180) to the sample plate (250) via convection.
   20. The system of clauses 13-19, wherein the adaptor plate (300) is configured to mate with a second sample plate, the second sample plate comprising one or more features different from one or more features of the first sample plate.
   21. The system of clauses 13-20, wherein the adaptor wall (320) comprises a plurality of slots (314), a slot of the plurality of slots configured to receive a rib (264) of the sample plate (250).
   22. The system of clauses 13-21, wherein the plurality of cavities (312) of the receptacle region (310) are arranged in a grid.
   23. The system of clauses 13-22, wherein a shape of the at least one cavity (312) comprises an inverted pyramid, a hemi-sphere, an inverted cone, or an inverted top-hat profile.
   24. The system of clauses 13-23, wherein the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.
   25. The system of clauses 13-24, wherein the adaptor plate (300) is formed by casting, stamping, forging, additive manufacturing, subtractive manufacturing, or a combination thereof.
   26. The system of clauses 13-25, the adaptor plate further comprising: a flange (304) disposed along a foot of the outer surface (324) of the adaptor wall (320), the flange configured to form the series of chambers.
   27. A method for coupling a sample plate (250) to a heater (180) via an adaptor plate (300), wherein the adaptor plate (300) comprises:
a receptacle region (310), the receptacle region comprising a plurality of cavities (312);
an adaptor wall (320) disposed along a perimeter of the receptacle region (310), the adaptor wall (320) comprising:
   an inner surface (326);
   a top edge (322); and
   an outer surface (324) connected to the inner surface (326) by the top edge (322); the method comprising:
coupling the adaptor plate (300) to the heater (180); and
placing the sample plate (250) on the adaptor plate (300), such that:
   at least one cavity of the plurality of cavities (312) mates with a corresponding well (252) of the sample plate (250),
   the inner surface (326) of the adaptor wall (320) mates with outer surfaces of one or more wells (252) of the sample plate (250), and
   the top edge (322) and the outer surface (324) of the adaptor wall (320) and a housing (260) of the sample plate (250) form a series of chambers when the adaptor plate (300) is mated with the sample plate (250).
      28. The method of clause 27, wherein coupling the adaptor plate (300) to the heater (180) comprises:
placing the adaptor plate (300) on the heater (180);
inserting a fastener through an aperture on the adaptor plate (300) and into a corresponding aperture disposed on the heater (180); and
and securing the adaptor plate (300) to the heater (180) with the fastener.
   29. The method of clause 27 or 28, further comprising disposing at least one of a thermal paste and a thermal transfer fluid between the adaptor plate (300) and the sample plate (250).
   30. The method of clauses 27-29, further comprising disposing at least one of a thermal paste and a thermal transfer fluid between the adaptor plate (300) and the heater (180).
   31. The method of clauses 27-30, wherein the sample plate (250) comprises:
the housing (260) having an upper surface (266) and an outer wall (262);
a well-region, the well-region comprising a plurality of wells (252) disposed within the housing (260), wherein the well (252) of the plurality of wells (252) comprises:
   an opening located at the upper surface (266) of the housing (260);
   a sidewall (254) extending from the opening into the housing (260);
   a bottom well-surface (258), the bottom well-surface (258) configured to mate with the at least one cavity (312) of the adaptor plate (300); and
a plurality of ribs (264), the plurality of ribs (264) disposed within the housing (260) and extending from the sample-well region to the outer wall (262).
   32. The method of clauses 27-31, wherein the heater comprises a thermoshaker.
   33. The method of clauses 27-32, wherein the adaptor plate (300) is configured to form a first gap between the top edge (322) of the adaptor wall (320) and the housing (260) of the sample plate (250) when the adaptor plate (300) is mated with the sample plate (250).
   34. The method of clauses 27-33, wherein the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap.
   35. The method of clauses 27-34, wherein, the adaptor wall (320) is configured to transfer heat from the heater (180) to the sample plate (250) via conduction.
   36. The method of clauses 27-35, wherein, fluid disposed in the series of chambers is configured to transfer heat from the heater (180) to the sample plate (250) via convection.
   37. The method of clauses 27-36, wherein the adaptor plate (300) is configured to mate with a second sample plate, the second sample plate comprising one or more features different from one or more features of the first sample plate.
   38. The method of clauses 27-37, wherein the adaptor wall (320) comprises a plurality of slots (314), a slot of the plurality of slots configured to receive a rib (264) of the sample plate (250).
   39. The method of clauses 27-38, wherein the plurality of cavities (312) of the receptacle region (310) are arranged in a grid.
   40. The method of clauses 27-39, wherein a shape of the at least one cavity (312) comprises an inverted pyramid, a hemi-sphere, an inverted cone, or an inverted top-hat profile.
   41. The method of clauses 27-40, wherein the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.
   42. The method of clauses 27-41, wherein the adaptor plate (300) is formed by casting, stamping, forging, additive manufacturing, subtractive manufacturing, or a combination thereof.
   43. The method of clauses 27-42, the adaptor plate further comprising: a flange (304) disposed along a foot of the outer surface (324) of the adaptor wall (320), the flange configured to form the series of chambers.
   44. A method for heating a sample, the method comprising:
loading the sample into a plurality of wells (252) of a sample plate (250);
mounting an adaptor plate (300) to a heater (180), wherein the adaptor plate (300) comprises:
   a receptacle region (310), the receptacle region comprising a plurality of cavities (312);
   an adaptor wall (320) disposed along a perimeter of the receptacle region (310), the adaptor wall (320) comprising:
      an inner surface (326);
      an outer surface (324); and
      a top edge (322) connecting the outer surface (324) to the inner surface (326);
placing the sample plate (250) on the adaptor plate (300) such that:
   at least one cavity of the plurality of cavities (312) mates with a corresponding well (252) of the sample plate (250),
   the inner surface (326) of the adaptor wall (320) mates with outer surfaces of one or more wells (252) of the sample plate (250), and
   the top edge (322) and the outer surface (324) of the adaptor wall and a housing (260) of the sample plate (250) form a series of chambers when the adaptor plate (300) is mated with the sample plate (250);
operating the heater at a first temperature for a first time period; and
operating the heater at a second temperature for a second time period.
   45. The method of clause 44, further comprising heating the sample plate (250) by conduction, via the adaptor wall (320) and further via the plurality of cavities (312) of the adaptor plate (300).
   46. The method of clause 45, further comprising heating the sample plate (250), by convection, via a fluid disposed in the series of chambers.
   47. The method of clause 46, wherein the combination of convective and conductive heating provides a uniform temperature distribution of the sample disposed in the plurality of wells (252).
   48. The method of clause 47, wherein the uniform temperature distribution comprises a temperature deviation between wells of the plurality of wells (252) of about two degrees centigrade.
   49. The method of clauses 44-48, wherein the sample plate (250) comprises:
the housing (260) having an upper surface (266) and an outer wall (262);
a well-region, the well-region comprising a plurality of wells (252) disposed within the housing (260), wherein the well (252) of the plurality of wells (252) comprises:
   an opening located at the upper surface (266) of the housing (260);
   a sidewall (254) extending from the opening into the housing (260);
   a bottom well-surface (258), the bottom well-surface (258) configured to mate with the at least one cavity (312) of the adaptor plate (300); and
a plurality of ribs (264), the plurality of ribs (264) disposed within the housing (260) and extending from the sample-well region to the outer wall (262).
   50. The method of clauses 44-49, wherein the heater comprises a thermoshaker.
   51. The method of clauses 44-50, wherein the adaptor plate (300) is configured to form a first gap between the top edge (322) of the adaptor wall (320) and the housing (260) of the sample plate (250) when the adaptor plate (300) is mated with the sample plate (250).
   52. The method of clauses 44-51, wherein the adaptor plate (300) is configured to form a second gap between the outer surface (324) of the adaptor wall (320) and an outer wall (262) of the housing (260) of the sample plate (250) when the adaptor plate (300) is mated with the sample plate (250), the second gap different than the first gap.
   53. The method of clauses 44-52, wherein, the adaptor wall (320) is configured to transfer heat from the heater (180) to the sample plate (250) via conduction.
   54. The method of clauses 44-53, wherein, fluid disposed in the series of chambers is configured to transfer heat from the heater (180) to the sample plate (250) via convection.
   55. The method of clauses 44-54, wherein the adaptor plate (300) is configured to mate with a second sample plate, the second sample plate comprising one or more features different from one or more features of the first sample plate.
   56. The method of clauses 44-55, wherein the adaptor wall (320) comprises a plurality of slots (314), a slot of the plurality of slots configured to receive a rib (264) of the sample plate (250).
   57. The method of clauses 44-56, wherein the plurality of cavities (312) of the receptacle region (310) are arranged in a grid.
   58. The method of clauses 44-57, wherein a shape of the at least one cavity (312) comprises an inverted pyramid, a hemi-sphere, an inverted cone, or an inverted top-hat profile.
   59. The method of clauses 44-58, wherein the adaptor plate (300) is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.
   60. The method of clauses 44-59, wherein the adaptor plate (300) is formed by casting, stamping or forging, additive manufacturing, subtractive manufacturing or a combination thereof.
   61. The method of clauses 44-60, the adaptor plate further comprising: a flange (304) disposed along a foot of the outer surface (324) of the adaptor wall (320), the flange configured to form the series of chambers.
   62. A method for measuring a temperature of wells (252) of a sample plate (250), the method comprising:
loading a sample into a plurality of wells (252) of the sample plate (250);
mounting an adaptor plate (300) to a heater (180), wherein the adaptor plate comprises:
   a receptacle region (310), the receptacle region comprising a plurality of cavities (312);
   an adaptor wall (320) disposed along a perimeter of the receptacle region (310), the adaptor wall (320) comprising:
      an inner surface (326);
      an outer surface (324); and
      a top edge (322) connecting the outer surface (324) to the inner surface (326);
placing the sample plate (250) on the adaptor plate (300) such that:
   at least one cavity of the plurality of cavities (312) mates with a corresponding well (252) of the sample plate (250),
   the inner surface (326) of the adaptor wall (320) mates with outer surfaces of one or more wells (252) of the sample plate (250), and
   the top edge (322) and the outer surface (324) of the adaptor wall and a housing (260) of the sample plate (250) form a series of chambers when the adaptor plate (300) is mated with the sample plate (250);
disposing a temperature sensor in a first well (252) of the sample plate;
sealing the plurality of wells;
operating the heater at a first temperature for a first time period;
operating the heater at a second temperature for a second time period; and
receiving measurements from the temperature sensor during the first time period and the second time period.
   63. The method of clause 62, further comprising disposing a second temperature sensor in a second well of the sample plate (250).
   64. A DNA extractor comprising:
a sample plate (250);
an adaptor plate (300) configured to mate with the sample plate (250), the adaptor plate (300) comprising:
   a receptacle region (310), the receptacle region comprising a plurality of cavities, at least one cavity (312) of the plurality of cavities configured to mate with a corresponding well (252) of the sample plate (250);
an adaptor wall (320) disposed along a perimeter of the receptacle region (310), the adaptor wall (320) comprising:
   an inner surface (326) configured to contact outer surfaces of one or more wells (252) of the sample plate (250);
   an outer surface (324); and
   a top edge (322) connecting the outer surface (324) to the inner surface (326), wherein the top edge (322) and the outer surface (324) of the adaptor wall and a housing (260) of the sample plate (250) are configured to form a series of chambers when the adaptor plate (300) is mated with the sample plate (250); and
   a bottom surface (306); and
a heater (180) configured to mate with the bottom surface (306) of the adaptor plate (300).
   65. A method for extracting deoxyribonucleic acid (DNA) molecules from a sample from a subject using a DNA extraction device, the method comprising:
providing the sample in a sample plate (250);
mounting an adaptor plate (300) to a heater (180), wherein the adaptor plate comprises:
   a receptacle region (310), the receptacle region comprising a plurality of cavities (312);
   an adaptor wall (320) disposed along a perimeter of the receptacle region (310), the adaptor wall (320) comprising:
      an inner surface (326);
      an outer surface (324); and
      a top edge (322) connecting the outer surface (324) to the inner surface (326);
placing the sample plate (250) on the adaptor plate (300) such that:
   at least one cavity of the plurality of cavities (312) mates with a corresponding well (252) of the sample plate (250),
   the inner surface (326) of the adaptor wall (320) mates with outer surfaces of one or more wells (252) of the sample plate (250), and
   the top edge (322), the outer surface (324) of the adaptor wall, and a housing of the sample plate form a series of chambers when the adaptor plate (300) is mated with the sample plate (250);
heating, via the heater (180), the sample in the sample plate (250);
extracting a plurality of nucleic acid molecules, including at least one DNA molecule, from the sample in the sample plate (250);
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules; and
sequencing, by a sequencer, the captured nucleic acid molecules to obtain a plurality of sequence reads that represent the captured nucleic acid molecules, wherein one or more of the plurality of sequencing reads overlap one or more gene loci within a subgenomic interval in the sample.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. An adaptor plate configured to couple a sample plate to a heater, the adaptor plate comprising:
a receptacle region the receptacle region comprising a plurality of cavities, at least one cavity of the plurality of cavities configured to mate with a corresponding well of the sample plate
an adaptor wall disposed along a perimeter of the receptacle region , the adaptor wall comprising:
an inner surface configured to contact outer surfaces of one or more wells of the sample plate;
an outer surface and
a top edge connecting the outer surface to the inner surface wherein the top edge and the outer surface of the adaptor wall and a housing of the sample plate are configured to form a series of chambers when the adaptor plate is mated with the sample plate; and
a bottom surface configured to mate with the heater

2. The adaptor plate of claim 1, wherein the adaptor plate is configured to form a first gap between the top edge of the adaptor wall and the housing of the sample plate when the adaptor plate is mated with the sample plate.

3. The adaptor plate of claim 1, wherein the adaptor plate is configured to form a second gap between the outer surface of the adaptor wall and an outer wall of the housing of the sample plate when the adaptor plate is mated with the sample plate, the second gap different than the first gap.

4. The adaptor plate of claim 1, wherein the adaptor wall is configured to transfer heat from the heater to the sample plate via conduction.

5. The adaptor plate of claim 1, wherein, fluid disposed in the series of chambers is configured to transfer heat from the heater to the sample plate via convection.

6. The adaptor plate of claim 1, wherein the adaptor plate is configured to mate with a second sample plate, the second sample plate different from the first sample plate.

7. The adaptor plate of claim 1, wherein the adaptor wall comprises a plurality of slots, a slot of the plurality of slots configured to receive a rib of the sample plate

8. The adaptor plate of claim 1, wherein the adaptor plate is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.

9. The adaptor plate of claim 1, the adaptor plate further comprising:
a flange disposed along a foot of the outer surface of the adaptor wall, the flange configured to form the series of chambers.

10. A system comprising:
a sample plate
an adaptor plate configured to mate with the sample plate, the adaptor plate comprising:
a receptacle region, the receptacle region comprising a plurality of cavities, at least one cavity of the plurality of cavities configured to mate with at least one corresponding well of the sample plate;
an adaptor wall disposed along a perimeter of the receptacle region, the adaptor wall comprising:
an inner surface configured to contact outer surfaces of one or more wells of the sample plate;
an outer surface; and
a top edge connecting the outer surface to the inner surface, wherein the top edge and the outer surface of the adaptor wall and a housing of the sample plate are configured to form a series of chambers when the adaptor plate is mated with the sample plate; and
a bottom surface; and
a heater configured to mate with the bottom surface of the adaptor plate.

11. The system of claim 10, wherein the sample plate comprises:
the housing having an upper surface and an outer wall;
a well-region, the well-region comprising a plurality of wells disposed within the housing, wherein the at least one corresponding well of the plurality of wells comprises:
an opening located at the upper surface of the housing;
a sidewall extending from the opening into the housing;
a bottom well-surface the bottom well-surface configured to mate with the at least cavity of the adaptor plate and
a plurality of ribs, the plurality of ribs disposed within the housing and extending from the sample-well region to the outer wall.

12. The system of claim 10, wherein the heater comprises a thermoshaker.

13. The system of claim 10, wherein the adaptor plate is configured to form a first gap between the top edge of the adaptor wall and the housing of the sample plate when the adaptor plate is mated with the sample plate.

14. The system of claim 10, wherein the adaptor plate is configured to form a second gap between the outer surface of the adaptor wall and an outer wall of the housing of the sample plate when the adaptor plate is mated with the sample plate the second gap different than the first gap.

15. The system of claim 10, wherein, the adaptor wall is configured to transfer heat from the heater to the sample plate via conduction.

16. The system of claim 10, wherein, fluid disposed in the series of chambers is configured to transfer heat from the heater to the sample plate via convection.

17. The system of claim 10, wherein the adaptor plate is configured to mate with a second sample plate, the second sample plate comprising one or more features different from one or more features of the first sample plate.

18. The system of claim 10, wherein the adaptor wall comprises a plurality of slots , a slot of the plurality of slots configured to receive a rib of the sample plate.

19. The system of claim 10, wherein the adaptor plate is formed from aluminum, copper, silver, gold, an aluminum alloy, a copper alloy, a gold alloy, a silver alloy, aluminum compounded with a binder, copper compounded with a binder, silver compounded with a binder, or gold compounded with a binder.

20. The system of claim 10, the adaptor plate further comprising: a flange disposed along a foot of the outer surface of the adaptor wall, the flange configured to form the series of chambers.

21. A method for heating a sample, the method comprising:
loading the sample into a plurality of wells of a sample plate;
mounting an adaptor plate to a heater, wherein the adaptor plate comprises:
a receptacle region, the receptacle region comprising a plurality of cavities;
an adaptor wall disposed along a perimeter of the receptacle region, the adaptor wall comprising:
an inner surface
an outer surface and
a top edge connecting the outer surface to the inner surface;
placing the sample plate on the adaptor plate such that:
at least one cavity of the plurality of cavities mates with a corresponding well of the sample plate,
the inner surface of the adaptor wall mates with outer surfaces of one or more wells of the sample plate, and
the top edge and the outer surface of the adaptor wall and a housing of the sample plate form a series of chambers when the adaptor plate is mated with the sample plate;
operating the heater at a first temperature for a first time period; and
operating the heater at a second temperature for a second time period.

22. The method of claim 21, further comprising heating the sample plate by conduction, via the adaptor wall and further via the plurality of cavities of the adaptor plate.

23. The method of claim 22, further comprising heating the sample plate by convection, via a fluid disposed in the series of chambers.

24. The method of claim 23, wherein the combination of convective and conductive heating provides a uniform temperature distribution of the sample disposed in the plurality of wells, and wherein the uniform temperature distribution comprises a temperature deviation between wells of the plurality of wells of about two degrees centigrade.
